SEQUENCE LISTING (32 sequences)

SEQ ID NO: 1 - protein, 189 aa, Plasmodium yoeli
MLMLLRRRCRAWLEIRRLDKELAQSSGLPLELPQIVPNAWNEVVWRLPVPNHPDAFMTASNAAQSDFIVYVNGLAFYRAWLALGVEDSQACPLKQDMPKDRKYPSSAAHFAVGIDSPVPLADVSPTMILGHFAVCFTDGMTRSMWLLAHEVAVFPVLSRDEASAVMLAEHVGVAAPIQVSKLREQCRKI

SEQ ID NO: 2 - DNA, 566 bp, Plasmodium yoeli
atgctgatgttgctacggcggcggtgtcgcgcttggctggaaattcggcggcttgataaagagcttgcgcagtcgagcgggttgcccctcgaattgcctcaaatcgtgccgaatgcttggaatgaggttgtttggcggctgcccgtgccgaaccatcctgacgcctttatgacagcttcaaatgccgcacagtccgatttcattgtgtatgtgaatggcttggctttctatcgagcgtggttggcgttaggcgtcgaggattctcaagcctgcccgttgaagcaggacatgcctaaagaccggaaatatccgtcgagcgccgcgcattttgccgtgggcatcgacagccccgttccgcttgctgacgtaagcccgaccatgattctgggccatttcgcggtgtgtttcactgatggcatgacccgttcaatgtggcttttggcccatgaggtcgccgtgtttccggtgctttcccgtgatgaagcatccgccgttatgttggcagaacacgtgggcgtagcagcgccgattcaggtttcaaagctgcgtgagcaatgcaggaagat

SEQ ID NO: 3 - DNA, 606 bp, Plasmodium yoeli
atgaccgacacgcgccgcgagcaggagaaagacgaacgccggaagctgcaagagcagtcgcgccagaatgaagcggaaaccatgcgcctgctggctttcgaggcaggccgccaattggccgaaattccgaaagaagccaaaggcaatgaacccttgcttgagaactacaagagcggactacaagagacccgcaaagagctagaaaccacgccagacgccactaagagcaccaacgccaaccggcttgagcgcgacgtagaaagggccatcatcgaggcgcaacaggtgcgcgaggcagtaggcagggagaaagcccgcgcagatgaatttcaccgccacgcagaaccgggcgaaacttaccggggccgtgtgatcggtcgcaccaatagctatgtcattcaggccgacgatagccgcccaggcacgatcattctgcatgaacgcgccgctgtttcgggcgcggagaaagtgaagatgaacgaccatgcggaaatcagttatccgcacggtcgcgccggaatcgtccgcaacccacaggccgcgcaacatcaacgacagcggcagatggaaaaaaccggcgcaggccgagagcatgggcgctaacga

SEQ ID NO: 4 - protein, 200 aa, Plasmodium yoeli
MTDTRREQEKDERRKLQEQSRQNEAETMRLLAFEAGRQLAEIPKEAKGNQPLLENYKSGLQETRKELETTPDATKSTNANRLQRDVERAIIEAQQVRQAVGREKARADQFHRHAEPGETYRGRVIGRTNSYVIQADDSRPGTIILHQRAAVSGAEKVKMNDHAQISYPHGRAGIVRNPQAAQHQRQRQMQKTGAGREHGR

SEQ ID NO: 5 - DNA, 563 bp, Piasmodium yoeli
ctagggcgtgtccctgcgctttgctccgggccgtgctctattccgtttgacctggccgccgctgcgctctggctaggtcaaactccaccgaacccccttcggggtttcggcccattcgggtaacgatcactcacgcctccgggccgctgtagcgtccctgcgcgcccctatcggggcttgccatcccttcgcaccctgccccggctcatcatccctattcggccgctcacgcccttcaagctgccctacggctcggcttgtccgggcaagggtacgcttcgccgccatcctcacccgttcggtgctcgcctttggctgcgctccgcgtgcggcttccggtggcgcccttgcccgcgcctcccctgcgggcagatcgggcttaccgcgacgaacgacgaatgacgagcagaccccgaggcacccggtttcatgctccggggccatgaagatgcgccgcctatcggcggcagatcaaacccatgaagggcgctaaagcgccctattcgtcggccttcggccgccgcccgctcccttcactcggaaatcaaagcaggcccggaaaa

SEQ ID NO: 6 - protein, 20 aa, Plasmodium yoeii
PVLSRDEASAVMLAEHVGVA

SEQ ID NO: 7 - protein, 18 aa, Saccharomyces cerevisiae
LRISTPYNAKHIHHVGVD

SEQ ID NO: 8 - protein, 19 aa, Homo sapiens
DMISHPLGDFRHTMHVGRG

SEQ ID NO: 9 - protein, 18 aa, Homo sapiens
ADIGAPSGFKHVSHVGWD

SEQ ID NO: 10 - protein, 18 aa, Schizosaccharomyces pombe
TVISSPFDPKHVTHVGFN

SEQ ID NO: 11 - protein, 18 aa, Rattus rattus
PEISLPSDFEHTIHVGFD

SEQ ID NO: 12 - protein, 18 aa, Caenorhabditis elegans
PVISRPSNFEHTIHVGYD

SEQ ID NO: 13 - DNA, 25 bp, Transposon Tnl000
cctgaaaagggacctttgtatactg

SEQ ID NO: 14 - DNA, 19 bp, Transposon Tnl000
aggggaactgagagctcta

SEQ ID NO: 15 - DNA, 18 bp, Plasmodium yoeli
atgctgatgttgctacgg

SEQ ID NO: 16 - DNA, 21 bp, Plasmodium yoeli
atcttcctgcattgctcacgc

SEQ ID NO: 17 - DNA, 19 bp, Plasmodium yoeli
cttggaatgaggttgtttg

SEQ ID NO: 18 - DNA, 18 bp, Plasmodium yoeli
aatcctcgacgcctaacg

SEQ ID NO: 19 - DNA, 28 bp, Homo sapiens
caggaattccagacaattaagtgtgttg

SEQ ID NO: 20 - DNA, 29 bp, Homo sapiens
caggtcgacttagaatatacagcacttcc

SEQ ID NO: 21 - DNA, 27 bp, Homo sapiens
caggaattccaggccatcaagtgtgtg

SEQ ID NO: 22 - DNA, 28 bp, Homo sapiens
caggtcgacctacaacaggcattttctc

SEQ ID NO: 23 - DNA, 30 bp, Homo sapiens
caggaattcgctgccatccggaagaaactg

SEQ ID NO: 24 - DNA, 28 bp, Homo sapiens
cagcgtcgactcacaagacaaggcaacc

SEQ ID NO: 25 - DNA, 26 bp, Plasmodium yoeli
cagggaattcatgctgatgttgctac

SEQ ID NO: 26 - DNA, 24 bp, Plasmodium yoeli
cagcgtcgacctagatcttcctgc

SEQ ID NO: 27 - DNA, 29 bp, Plasmodium yoeli
catgctcgagatcttcctgcattgctcac

SEQ ID NO: 28 - DNA, 588 bp, Cryptosporidium parvum
cgagaattcatgctaatgttgctacggcggcggtgtcgcgcttggctggaaattcggcggcttgataaagagcttgcgcagtcgagcgggttgcccctcgaattgcctcaaatcgtgccgaatgcttggaatgaggttgtttggcggctgcccgtgccgaaccatcctgacgcctttatgacagcttcaaatgccgcacagtccgatttcattgtgtatgtgaatggcttggctttctatcgagcgtggttggcgttaggcgtcgaggattctcaagcctgcccgttgaagcaggacatgcctaaagaccggaaatatccgtcgagcgccgcgcattttgccgtgggcatcgacagccccgttccgcttgctgacgtaagcccgaccatgattctgggccatttcgcggtgtgtttcactgatggcatgacccgttcaatgtggcttttggcccatgaggtcgccgtgtttccggtgctttcccgtgatgaagcatccgccgttatgttggcagaacacgtgggctgtagcagcgccgattcaggtttcaaagctgcgtgagcaatgcaggaagatctagaagcttcg

SEQ ID NO: 29 - DNA, 102 bp, Plasmodium falciparum
  misc_feature 28: Primer sequence
gangctttatttttgcttcaaatgcgcncagtccgatttcattgtgtatgtgaatggcttggctttctatcgagcgtggttggcgttaggcgtcgaggatta

SEQ ID NO: 30 - DNA, 98 bp, Plasmodium falciparum
gcttatgacagcttcaaatgcgcacagtccgatttcattgtgtatgtgaatggcttggctttctatcgagcgtggttggcgttaggcgtcgaggatta

SEQ ID NO: 31 - DNA, 150 bp, Plasmodium yoeli
gaaccatcctgacgcctttatgacagcttcaaatgccgcacagtccgatttcattgtgtatgtgaatggcttggctttctatcgagcgtggttggcgttaggcgtcgaggattctcaagcctgcccgttgaagcaggacatgcctaaaga

SEQ ID NO: 32 - DNA, 95 bp, Plasmodium falciparum
atgcctttatgactgcttcaatgcgccagccgatttcattgtgtatgtgaatggcttggcttctatcgagcgtggttggcgttaggcgtcgagga

(19) United States
(12) Patent Application Publication
(10) Pub. No.: US 2005/0260224 A1
(43) Pub. Date: Nov. 24, 2005

(54) Vaccine based on a cellular penetration factor from an apicomplexan parasite

(76) Inventors: Stephen Henry Gillespie, London (GB); Henry Kean Bayele, London (GB); Timothy Daniel McHugh, London (GB)

Correspondence Address: Pillsbury Winthrop;Intellectual Property Group, PO Box 10500, McLean, VA, 22102, US

(21) Appl. No.: 10/416,384

(22) PCT Filed: Nov. 9, 2001
(86) PCT No.: PCT/GB01/04985

§ 371 Date: Nov. 3, 2003

(30) Foreign Application Priority Data
Nov. 9, 2000 (GB) 0027433.2

(51) Int. Cl. A61K 39/002

(52) U.S. Cl. 424/191.1

(57) ABSTRACT

An antigenic component, for use in a vaccine capable of promoting, production, in a subject of an antibody specific to the antigenic component, which antibody is capable of specifically binding to the Pid protein having the amino acid sequence in Seq ID No. 1.

This application is the National Phase of International Application PCT/GB01/04985 filed Nov. 9, 2001 which designated the U.S. and that International Application was published under PCT Article 21(2) in English.

### FIELD OF THE INVENTION

This invention relates to an antigenic component for use in a vaccine, particularly for diseases such as those caused by apicomplexan parasites.

### BACKGROUND TO THE INVENTION

The apicomplexans comprise a range of parasites including those of the genera: Eimeria; Isospora; Toxoplasma; Hammondia; Cystoisospora; Sarcocystis; Besnoitia; Frenkelia; Cryptosporidia; Plasmodia; Babesia; and Theileria.

All genera of the apicomplexans have a specialized organelle called the apical complex (hence their name). This organelle contains secretory granules/proteins that are extruded onto the surface of target cells during invasion. Extrusion of these proteins precedes cell entry

These parasites are associated with disease in a wide variety of host organisms. Eimeria species, for example, are known to be pathogenic to at least chickens, turkeys, geese, ducks, cattle, sheep, pigs, horses, rabbits, rats and mice. Toxoplasmosis of humans, puppies and lambs is associated with Toxoplasma species, in particular, T. gondii. Cryptosporidia species infect mammals, birds and reptiles. C. muris and C. parvum in particular are known to cause gastrointestinal disease in cattle, sheep and humans.

Babesiosis, associated with Babesia species, is an often fatal disease of domesticated animals, including cattle, horses, sheep, goats, pigs, cats and dogs. Theileria species are known to infect cattle, sheep and goats. T. parva and T. annulata in particular, are important pathogens in cattle, the former causing African theileriosis or East Coast Fever.

One of the most intensively studied apicomplexan associated diseases is malaria. The disease is today one of the most significant single causes of human morbidity and mortality, with estimated death rates of up to 3 million and approximately 500 million infected cases per year (Butler, D. and J. Maurice, 1997).

Malaria is caused by Plasmodium species which are injected into the blood of vertebrates by female mosquito vectors. To date, four Plasmodium species have been associated with human malaria: P. falciparum; P. vivax; P. ovate; and P. malariae. Of these, P. falciparum is believed to be the major cause worldwide. Additionally, there are known to be at least 20 species of Plasmodium in non-human primates, including: P. cynomolgi; P. knowlesi; P. brasilianum; P. inui; P. berghei; P. yoelii; P. vinckei; and P. chabaudi.

The lifecycle of Plasmodium spp. is illustrated in Figure 0.1.

The infective stage, the sporozoites, are injected directly into the bloodstream from the salivary glands of a mosquito. These sporozoites then invade liver cells, within which they replicate in a process referred to as extra-erythrocytic schizogony. At this stage, some P. vivax or P. ovale parasites will develop into hypnozoites, which remain dormant, but which, upon reactivation, may cause relapses.

After a (species-dependent) period of time, the parasites, now called merozoites, reinvade the circulation. The merozoites invade red blood cells, and undergo a further phase of replication, referred to as erythrocytic schizogony. Following rupture of the infected red blood cells, the released merozoites may in turn invade new red blood cells. This cycle of infection may be repeated many times.

The merozoites are believed to be the primary cause of malarial pathology. For example, the parasites provoke the release of cytokines, such as tumour necrosis factor, whose action is thought to be responsible for many of the signs and symptoms of malaria. Furthermore, cerebral malaria is known to result from infected red blood cells adhering to capillaries in the brain.

Some of the merozoites are capable of developing into the sexual stages or gametocytes, which are taken up when a female mosquito bites again. After a period of fertilisation, ookinetes are formed and invade the mosquito gut, in preparation for development into sporozoites. The sporozoites in turn penetrate the mosquito salivary glands, in readiness for the next bite.

Current treatments against diseases associated with apicomplexan parasites other than malaria have met with limited success. There is only one vaccine available for this group of organisms, a live attenuated vaccine for Toxoplasmosis that is only licenced for animal use. Treatment with folate inhibitors and macrolides is available for toxoplasmosis, but there is a need to develop new treatments for use during pregnancy as the most effective treatment, sulphadiazine and pyrimethamine, is unsafe during pregnancy. Macrolides such as spiramycin, although safe are less effective and are unable to cross the blood-brain barrier, and thus is unsuitable for the treatment of cerebral toxoplasmosis. There is currently no treatment or vaccine available for cryptosporidiosis.

Anti-disease strategies for malaria broadly include mosquito bite prevention, anti-parasitic drugs and prophylactic treatment.

Attempts have been made since the 1980s to use protein components of P. falciparum to develop vaccines which would stimulate the production of protective antibodies in a host. However, none of the vaccines tested have provoked a strong enough immune response to be effective in the field. SPf66, a composite vaccine targeting the blood stage, appeared to provide some protection in field trials, but at present is not thought to be a suitable candidate for malarial control.

SPf66 was found to be immunogenic and to provide some level of protection (30-35%) in South American volunteers, but was largely unprotective in African children who are exposed to higher levels of infectivity. This means that the efficacy of this vaccine is not strain-transcending.

For any vaccine/drug to be effective, it must cross parasite-strain boundaries regardless of the geography of disease prevalence. X-ray irradiated sporozoites have been shown to be effective in a challenge study but impractical for widespread use. A vaccine using the major sporozoite protein, the circumsporozoite protein, CSP, did not produce long-lasting immunity as it did not induce T- cell responses. Another vaccine based on the merozoite surface protein, MSP-1, failed to confer protection in monkey trials. The reason why all these vaccines fail is most probably due to a wrong choice of vaccine candidates derived directly from wrong premises. Another possible problem is that these antigens are highly polymorphic from one geographical region to the other; some are also redundant, i.e they occur in more than one copy (such as MSP-1) in the parasite. Crucially, the rationale for the choice of vaccine candidates does not take into account the biochemistry or biology of the cognate molecule and how it fits into the whole schema of parasite infectivity; for example, the functions of MSP-1 or CSP which have been widely studied over the last 20 yrs, have not been elucidated; prior knowledge of function of a molecule is an important prerequisite for its use in vaccine design.

Recently, attempts have been made to develop vaccines which will elicit a cell-mediated immune response in the host.

One such study has aimed to produce a vaccine that would stimulate host T-cells to destroy parasite-infected liver cells. The study has made use of a so-called “prime-boost” technique, in which the host immune system is primed with one vaccine and boosted with another, to increase the levels of cytotoxic T-cells. The two components of the prime-boost vaccine are: a DNA vaccine based on particular identified antigens; and a non-replicating vaccinia virus (MVA) having the gene for those same antigens inserted in its DNA. The prime-boost vaccine has been shown to provide protection against later malarial infection in mice. Human trials are currently underway.

In a further example, RTSS is a viral vaccine based on sporozoite protein which is currently in field trials.

However, despite this apparent progress there is as yet no effective anti-malarial vaccine. This being so, there remains a need for new treatments, both therapeutic and prophylactic, effective against malaria and other diseases associated with apicomplexan parasites.

### SUMMARY OF THE INVENTION

Accordingly in a first aspect the present invention provides an antigenic component, for use in a vaccine capable of promoting in a subject production of an antibody specific to the antigenic component, which antibody is capable of specifically binding to the Pid protein having the amino acid sequence in Seq ID No 1.

The present inventors have identified the pid (Plasmodium invasion determinant) locus as an invasiveness-conferring locus, occurring in apicomplexan parasites. A parasite-infected host would be expected to bear the Pid protein and antibodies to the protein, as a marker of infection. The Pid protein therefore provides a potential new target for treatments against diseases associated with these parasites, including vaccines and therapeutic agents.

Surprisingly, the newly identified Pid protein has been found to have an identical amino acid sequence to that of the Osa (oncogenic suppressive activity) protein, encoded by the osa gene of the pSa plasmid (Kado, C. I. and S. M. Close, 1991; Chen, CY and C. I Kado, 1994). The osa and pid loci also have identical nucleotide sequences.

In the field of plant pathology, the pSa plasmid is known to inhibit completely the ability of Agrobacterium tumefaciens to incite tumours in plants. The above referenced studies reported that the osa locus alone is sufficient for this inhibition to occur. The oncogenicity of A. tumefaciens is mediated by the transfer of a specific sector (T-DNA) of the bacterial Ti plasmid to the plant cell. The above studies suggested that the Osa protein might suppress oncogenicity by blocking the transfer or VirE2 from bacterium to plant.

According to the first aspect of the present invention; the Pid protein provides a specific target for antibodies raised in a subject in response to a vaccine.

In general, an antibody binds to a part of a protein known as an epitope or antigenic determinant. A protein may have more than one epitope, and different epitopes on one protein may be recognised by different antibodies. Similarly, a single antibody may be capable of binding to more than one epitope; however the affinity of binding, and so the specificity of the interaction, will vary.

The binding specificity of the antibodies raised in response to a vaccine is determined by the antigenic component of the vaccine. Briefly, on administration of the vaccine, the antigenic component is recognised by the host immune system, which produces antibodies capable of specifically binding to the component. In this context, binding between an antigenic component and a specific cognate antibody is expected to occur with a binding constant in the range 10⁻⁶-6 to 10⁻⁷ M or even lower.

In, the present case, the antibodies raised in response to the vaccine must also be capable of recognising and binding to the target Pid protein in the infected host. In order to avoid cross-reactivity with native host antigens., the antibodies must bind Pid specifically, typically with a binding constant in the range 1 to 10 nM and preferably below 1 nM.

In one embodiment, the antigenic component may comprise the Pid protein having the amino acid sequence in SEQ ID No. 1, or a variant thereof which does not substantially affect its antigenicity. In this way, antibodies, raised to bind specifically to an epitope of the Pid protein in the antigenic component, can bind that same epitope in the target Pid protein.

The Pid protein with a variant sequence may be a naturally occurring variant, or may be engineered. The variant sequence may comprise one or more amino acid additions, substitutions or deletions compared to the sequence in SEQ ID No 1. Similarly the variant may comprise one or more modified amino acids, provided that the variations in the amino acid sequence do not substantially affect the aritigenicity of the protein. For example, variation by conservative substitution is a possibility. Combinations of conservative substitutions are asparagine and glutamine (N or Q); valine, V, leucine, L, isoleucine, I, and methionine, M; aspartic acid and glutamic acid (D or E); lysine, K, arginine, R, and histidine, H); alanine, A, and glycine, G; serine, S, and threonine, T; phenylalanine, F, tyrosine, Y, and tryptophan, W.

Use of a variant Pid protein may provide particular advantages. The variant may, for example, have improved solubility or stability, or may be more compatible with other vaccine components. For example, a fusion tag such as thioredoxin may be linked to the protein to improve stability and/or solubility.

As explained above, a protein may comprise more than one epitope for antibody binding. Any epitope of the Pid protein may therefore be contained in only a fragment of the protein. Accordingly, the antigenic component may comprise a peptide fragment of the Pid protein having the amino acid sequence in SEQ ID No 1 or a variant thereof as previously described.

Use of a peptide fragment rather than the entire protein provides a smaller antigenic component which may be more easily administered. Small fragments may also be produced more cheaply and more easily than the intact protein.

These can be readily synthesized by synthetic chemistry or by recombinant methods.

Preferably, the Pid protein or peptide fragment of the Pid protein in the antigenic component is preparable from an apicomplexan parasite. The protein may be expressed by, and isolated from the source parasite. Alternatively, the pid locus may be isolated from a parasite and expressed using standard cloning and expression techniques. Suitable apicomplexan parasites include those selected from the following genera: Eimeria; Isospora; Toxoplasma; Hammondia; Cystoisospora; Sarcocystis; Besnoitia; Frenkelia; Cryptosporidium; Babesia; Theileria; and, in particular, Plasmodium.

A protein purified from the parasite source is near-to-native (having any required post-translational modification such as myristylation or glycosylation), and therefore preferred as a source of antigenic component. However, these proteins are difficult to purify in sufficient quantities. Bacterial expression is guaranteed to yield large quantities of recombinant protein, but this may not be post-translationally modified. However, expression in yeast (Pichia pastoris, Saccharomyces cerevisiae or Schizosaccharomyces pombe) or baculovirus/insect cell system, ensures that the recombinant protein is appropriately modified.

It is also however envisaged that synthetic mimics of the Pid protein may be constructed which are suitable for use in the antigenic component.

In a vaccine aimed at eliciting an antibody response, immunogenicity is mediated by the vaccine immunogen. Accordingly, in a second aspect the present invention provides an immunogen comprising the antigenic component coupled to an immunogenic component.

The antigenic component may itself be immunogenic so that the antigenic component itself comprises the immunogenic component. However, it may be that the antigenic component is, for example, too small to be immunogenic to the host. In that case, it may be necessary to couple the antigenic component to a suitable carrier. To be effective, therefore, the isolated antigen preferably stimulates the host immune system in a manner and at a level similar to that elicited during biological infection. To enhance antigen presentation and immunogenicity, it may be coupled to haptens such as bovine serum albumin and keyhole limpet haemocyanin; viral particles or dendrimers. It may also be possible to engineer attenuated Salmonella strains to carry vaccines to be delivered orally as live vaccines. Salmonella is appropriate for this purpose because it induces both high antibody and cell-mediated immune responses.

In a further aspect, the present invention provides a vaccine comprising an immunogen and an adjuvant, which enhances the antibody response. Freund's complete adjuvant and Freund's incomplete adjuvant, for example, are suitable for use in non-human vaccines. Aluminium hydroxide and aluminium phosphate are adjuvants authorised for human use. Possible further adjuvants include liposomes, BCG, lipopolysaccharides, muramyl dipeptide derivatives, squalene, non-ionic hydrophobic block copolymer surfactants such as polyoxypropylene and polyoxyethylene copolymers, pluronic polyols, ethylene-vinyl acetate, cyclodextrins and polysialic acid.

In a further aspect the present invention provides a vaccine comprising a polynucleic acid, which encodes the antigenic component described above. The polynucleic acid may comprise, for example, DNA, RNA or a synthetic nucleic acid.

In one embodiment, the polynucleic, acid comprises the sequence in SEQ ID No 2.

Polynucleic acid vaccines are typically aimed at eliciting a cell-mediated immune response in a subject. A key feature of this type of vaccine is that the antigenic component encoded by the polynucleic acid of the vaccine is expressed in and displayed on the surface of a cell within the subject. These vaccines may be of particular use against diseases associated with cell-invasive parasites, since they mimic the natural situation where the antigen is intracellular.

The polynucleic acid of the present vaccine may further comprise sequence for efficient expression of the antigenic component. For example, such sequence may comprise a promoter sequence or encode a secretion signal.

Advantageously, the present vaccine also comprises a delivery means for delivery of the polynucleic acid to a subject. The vaccine may additionally comprise an adjuvant for enhancing the cell-mediated immune response in the subject.

The present polynucleic acid vaccine preferably takes either of two main forms: a naked vaccine or a live vaccine.

In the case of a naked vaccine, the polynucleic acid is administered to a subject, and by one of a number of alternative means, is delivered to a target host cell. The antigenic component encoded by the polynucleic acid is then expressed by the host cell.

The polynucleic acid of a naked vaccine may, for example, comprise a plasmid, bearing a eukaryotic promoter to direct efficient expression of the antigenic component in a target host cell. The promoter may be constitutive, for example, the generic CMV promoter or SV40 promoter. Alternatively, the promoter may be tissue specific. In one embodiment the promoter is a muscle specific promoter such as the MyoD, myosin or myogenin promoters. The significance of a muscle-specific promoter is that a polynucleic acid vaccine delivered by intramuscular injection can be expressed directly by muscle cells.

The liver may also be targeted for expression of antigens; a liver-specific promoter such as the albumin promoter may be used in combination with a secretory signal tagged onto the antigen open reading frame for expression and secretion.

The polynucleic acid may additionally comprise sequence encoding a secretion signal for the antigenic component, to ensure that during expression the component is secreted to the outer surface of the host cell. For example, the secretion signal may comprise the malE signal for bacterial expression; the honeybee melittin signal for baculovirus expression in insect cells (Sf9; Sf21); the α-factor for expression in yeast and the Igκ signal for expression in mammalian cells.

The polynucleic acid of the naked vaccine may further comprise immunostimulatory sequences which provide a suitable adjuvant. For example, unmethylated CpG sequences may be used for this purpose. CpG immunostimulatory sequences may also be combined with one or more other adjuvants indicated above such as complete or incomplete Freund's adjuvant.

For delivery to a subject, the polynucleic acid of the naked vaccine may be complexed with for example, liposomal vesicles or viral particles. In one embodiment, the vaccine is delivered by injection through the skin or muscle. In a further embodiment, the vaccine is delivered by nebulisation. Liposomes and viral particles act as carriers. No particular cell types are targeted except when tissue-specific expression is desired wherein the requisite promoter will be included on the delivered DNA sequence.

In the case of a live vaccine, the polynucleic acid is first transformed into a suitable strain of bacteria, so that the bacterial cells express the antigenic component on their cell surface. The expressing bacterial strain is then administered to the subject, so that the bacteria provide the required delivery means.

Bacterial strains suitable for this purpose include attenuated aro/auxotrophic mutants of Salmonella, Listeria and Corynebacterium, pseudotuberculosis. Viral vectors such as attenuated herpes simplex, BCG and adenoviruses can also be used.

The polynucleic acid of a live vaccine may comprise an expression vector, bearing a prokaryotic promoter to direct efficient expression of the antigenic component in the bacterium. Suitable promoters for bacterial expression include the tac, trc, BAD, T7 and P_{L}/trp promoters. The polynucleic acid may also encode a secretion signal, directing secretion of the component from the bacterial cell, thereby exposing the component to the host immune system. Examples of secretion signals include malE, ompT, pelB and bacteriophage fd gene III protein signal.

In a further embodiment the polynucleic acid encoding the antigenic component may be integrated into the bacterial chromosome. Integration would be expected to provide improved stability and increased expression levels.

Expression of the antigen will typically be driven by any one of the above generic promoters. Alternatively, it may be possible to use, for example, a Salmonella gene promoter. The secretion signal will be an integral part of any targeting recombinant vaccine vector, and therefore will be stably integrated into the bacterial chromosome.

In the case of the live vaccine, the use of an adjuvant may be optimal where, for example, Salmonella is used because Salmonella is able to induce secretory, humoral and cellular immunity.

Preferably, the vaccines described above are suitable for use in a human subject. These vaccines will, for example, comprise an adjuvant suitable for use in humans, such as those described above. Typically, these vaccines will be non-pyrogenic, non-inflammatory and non-necreotizing, as well as being protective against biological infection.

In one embodiment, the present vaccine is suitable for use against human malaria caused by P. falciparum, P. ovale, P. vivax, or P. malariae.

Without wishing to be bound by theory, the present vaccine may target Pid at one or more of the life-cycle stages of an apicomplexan parasite. In the case of a Plasmodium parasite, the vaccine may, for example, target a lifecycle stage which is invasive to mammalian liver cells or red blood cells. The target life-cycle stage may be the sporozoites which invade the liver; the merozoites which invade red blood cells or the ookinetes, which invade the mosquito gut wall during or after a blood meal to complete development to sporozoites.

In addition to providing a new target for a vaccine, the Pid protein also provides a basis for new therapies against infectious disease, such as apicomplexan-associated disease.

Accordingly, in a further aspect the present invention provides a therapeutic agent comprising a component which component is capable of competing with a protein having the amino acid sequence in Seq ID No. 1 in a specific binding assay.

It is envisaged that the Pid protein has a critical role in cell invasion by, for example, apicomplexan parasites. Without wishing to be bound by theory this is likely to occur by interaction of Pid with a receptor. A component which is capable of competing with Pid in an in vitro specific binding assay is likely to be capable of competing with Pid in vivo for receptor binding. The component can therefore be incorporated into a therapeutic agent aimed at blocking Pid-receptor interaction.

In one embodiment, Pid can be used in combinatorial phage display selection from a pool of random peptides without prior knowledge of its target receptor(s) or interactors. A library of random peptides of up to 15 amino acids may be constructed and displayed on a phage surface. Recombinant Pid protein is then immobilized on Petri dishes, blocked with BSA to occlude non-specific sites. The phage-encoded peptides are then incubated with Pid. After this step, non- specifically bound peptides/phage are washed off and specific phage are eluted, amplified in permissive bacterial hosts and the whole panning cycle is repeated. After 4-5 selections, the peptide sequences are determined; the affinity of binding may then be further optimized by site-directed mutagenesis. These peptides are then synthesized and used in binding assays. High affinity binding may be defined as those peptides with a dissociation constant in the 1-10 nM range. IN₅₀ may be determined by competitive ELISA. Typically, a value of 10⁻⁶ to 10⁻⁷ is deemed competitive binding.

In one embodiment, It is envisaged that a mimic of the Pid protein may be produced and incorporated into a therapeutic agent. Alternatively, the receptor for Pid may be identified and an inhibitor of the Pid-receptor interaction used as a component of the therapeutic agent.

Antibodies to a Pid receptor may also be therapeutic since they may block interaction with Pid, assuming that receptor antibody epitopes and binding sites for Pid are the same.

In a further aspect the invention provides a protein comprising the amino acid sequence in SEQ ID No 1 or a fragment thereof for use in medicine. The invention additionally provides a polynucleic acid encoding the protein or a fragment of the protein, for use in medicine.

The vaccine and the therapeutic agent, both according to the present invention, are suitable for use in methods of treatment against infectious diseases such as those caused by apicomplexan parasites.

This aspect of the present invention therefore advantageously provides the use of the above antigenic component for the manufacture of a medicament effective against a disease caused by an apicomplexan parasite.

According to the above, manufacture of a medicament effective against such a disease may comprise the use of a protein comprising the amino acid sequence in SEQ ID No 1 or a peptide fragment thereof, and/or the use of a polynucleic acid encoding such a protein or peptide fragment.

Diseases which may suitably be treated according to the present invention include those caused by apicomplexan parasites of the following genera: Eimeria; Isospora; Toxoplasma; Hammondia; Cystoisospora; Sarcocystis; Besnoitia; Frenkelia; Cryptosporidium; Plasmodium; Babesia; and Theileria.

Preferably, the disease is one selected from the following: malaria; coccidiosis; theileriosis; cryptosporidiosis; isosporiasis; blastocystosis; babesiosis; anaplasmosis; sarcosporidiosis; toxoplasmosis; and sarcosystosis.

In particular, the invention provides a means for preventing and treating malaria disease, associated with the Plasmodium parasite. Human malaria, associated with for example, P. falciparum, P. vivax, P. ovale and P. malariae, is a particularly important target.

As noted above, the Pid protein provides a convenient marker of infection by an organism bearing the pid locus, such as an apicomplexan parasite. Accordingly, in one aspect the present invention provides a diagnostic agent comprising an antibody, which antibody is capable of specifically binding to the Pid protein having the amino acid sequence in SEQ ID No 1.

In this context, the antibody is typically capable of binding to the Pid protein with a binding constant in the range from 10⁻⁶ to 10⁻⁷, The diagnostic agent preferably comprises a means for detecting antibody-Pid complexes. The antibody may, for example, be labelled using standard techniques with a fluorophore, a radiolabel, a marker enzyme or a ligand. Suitable fluorophores include fluorescein, rhodamine, Cy3, TRITC and phycoerythrin. Suitable marker enzymes include alkaline phosphatase, beta galactosidase and horse radish peroxidase. Suitable ligands include biotin and digoxigenin (DIG).

In a further aspect the invention provides the use of the diagnostic agent in a method of diagnosing disease caused by an apicomplexan parasite. The diagnostic agent may for example be used in vitro to detect the presence of the Pid protein in a sample taken from a subject.

An in vitro diagnostic test of infection based on the detection of pid antibodies in patient serum/plasma may involve ELISA or EIA which may be complemented with immunofluorescence microscopy. Any diagnostic agent involving pid should include positive controls (purified pid protein and the cognate reactive antibodies) as well substrates for the relevant label [eg. p-nitrophenyl phosphate (NPP) for alkaline phosphate (AP)-labelled secondary antibodies].

Infection may also be identified by detecting antibodies to a Pid protein in the serum of a subject. Accordingly in a further aspect the present invention provides a diagnostic agent comprising an antigenic component as described above.

The antigenic component will bind to any Pid antibodies present in the subject sera; binding can be detected by one of a number of standard labelling techniques.

The present invention further provides the use of the diagnostic agent in a method of diagnosing disease caused by an apicomplexan parasite.

In one embodiment, the antigenic component of the diagnostic agent may be immobilised onto ELISA plates, and incubated with subject sera. Antibodies to Pid in the sera may then be detected by antigen capture, with monoclonal antibodies to Pid being used as a positive control.

Other labeling and detection methods may include chromogenic methods [e.g. AP-NPP or nitrotetrazolium blue and 5-bromo-4-chloro-3-indolyl phosphate (BCIP)], fluorogenic (e.g. biotin and fluorescein-labelled streptavidin) or luminescence (e.g AP and CDP-Star). While ELISA or EIA methods may be used, it may also be possible to perform Western blots or fluorescence in situ hybridization. In some cases, detection may be enhanced by in situ polymerase chain reaction using fluorogenic nucleotides.

The immobilised antigenic component may be provided in a spot test or dip-stick method for field diagnosis. Hapten- conjugated (FITC, alkaline phosphatase etc) monoclonal antibodies to Pid will then be incubated with parasites and visualised directly by fluorescence microscopy.

In accordance with the above, the present invention provides an antibody, which is capable of specifically binding to the Pid protein having the amino acid sequence in SEQ ID No 1 for use in medicine.

The invention also provides the use of such an antibody for the manufacture of a diagnostic agent for diagnosis of a disease caused by an apicomplexan parasite.

In a further aspect, the invention provides the use of an antigenic component as described above, for the manufacture of a diagnostic agent for diagnosis of a disease caused by an apicomplexan parasite.

It will be appreciated that to manufacture the diagnostic agents described above may comprise the use of a protein comprising the amino acid sequence in SEQ ID No 1 or a fragment thereof, or the use of a polynucleic acid encoding the protein or peptide fragment.

A diagnostic agent may be provided in a kit typicaly comprising positive controls (purified pid protein and the cognate reactive antibodies) as well, substrates for the relevant label [eg. p-nitrophenyl phosphate (NPP) for alkaline phosphate (AP)-labelled secondary antibodies]. A detailed and fully referenced instruction manual and calibration information would normally be an integral part of the kit.

In a further aspect there is provided an in vitro method for diagnosing apicomplexan infection in a subject, which comprises:
(i) obtaining from the subject a nucleic acid containing sample; and
(ii) testing the sample for the presence of nucleic acid sequence characteristic of Pid. The nucleic acid sequence characteristic of Pid may be all or a part of the sequence encoding the Pid protein or may be nucleic acid sequence upstream or downstream of the Pid coding sequence. Conveniently, the nucleic acid containing sample is subjected to a step of amplification, such as by PCR, which is preferably specific to the Pid nucleic acid sequence. This may be achieved using appropriate primers, such as any of those set out in FIG. 5. Other primers unique to the target nucleic sequence may also be used.

Infection by any of the apicomplexans described above may be detected using this method. Where the infection is in humans, detection of plasmodium infection is particularly important in diagnosing malaria. This may be achieved using red blood cells as the sample source of nucleic acid.

The apicomplexan associated diseases which may be diagnosed according to the present invention include those described above.

### BRIEF DESCRIPTION OF THE DRAWINGS

The invention will now be described in more detail by way of example only with reference to the accompanying drawings in which

FIG. 1 illustrates the lifecycle of a Plasmodium parasite;

FIGS. 2a and 2b shows the results of an immunofluorescence invasion assay on E. coli strains transformed with cosmid clones of P. yoeli DNA;

FIG. 2c shows transmission electron micrographs of COS-7 cells which have been invaded by E. coli strains transformed with ‘Inv’ cosmid clones of P. yoeli DNA;

FIG. 3 provides a bar chart displaying the results of plate scoring of wild type cosmid clones (Invcos11WT and Invcos18WT) and Tn1000γδ-inserted clones (TM1 and TM5), rescued from COS-7 cells following invasion assays;

FIG. 4a shows a nucleotide sequence including the PID nucleotide sequence (SEQ ID:NO. 2) and associated amino acid sequence (SEQ ID:NO. 1) according to the invention;

FIG. 4b shows a schematic representation of PID and adjacent sequences; and

FIG. 5 shows a part of the Pid nucleotide sequence identifying primer sites for diagnostic assays;

FIG. 6 shows results of PCR amplification of Pid from patient samples; and

FIG. 7 shows Pid sequences amplified from patients.

### DETAILED DESCRIPTION OF THE INVENTION

### Examples

Isolation of the pid locus from Plasmodium yoeli.

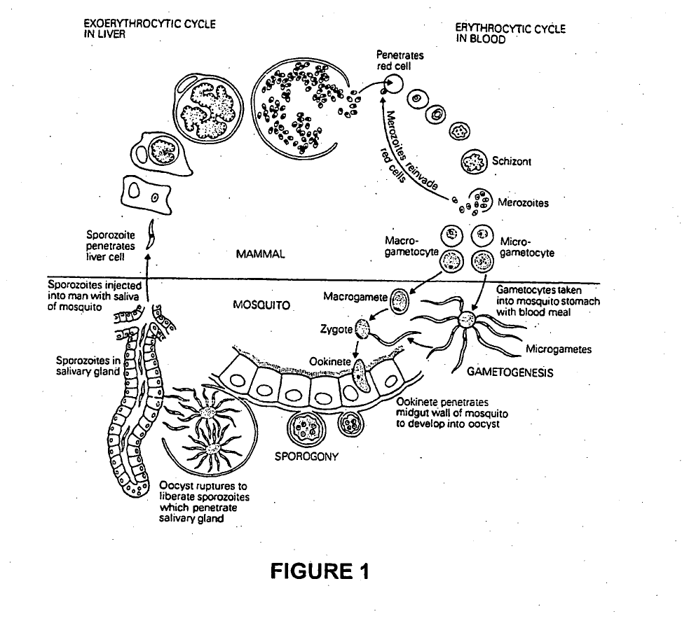

As described above, the lifecycle of Plasmodium sp. involves three invasive stages (FIG. 1): the ookinete; the sporozoite and the merozoite. Each stage has a different target cell preference. In the host, for example, sporozoites invade liver cells preferentially, whereas merozoites invade red blood cells.

In general terms there are three major steps in Plasmodium invasion: recognition, attachment and entry. The factor or factors which are involved in the invasive process have not been conclusively identified. A number of possible parasite specific ligands have been implicated in cell entry, and various cellular structures suggested as putative receptors. However these studies have focused on binding to the cell rather than on actual cell entry (Sim, B. K. L et al, 1994; Horuk, R et al, 1993; Breton, C. B et al, 1992; and Hadley, T J et al, 1986).

Using a reconstitution assay (Isberg, R. R. and S. Falkow, 1985), the present inventors have isolated from P. yoeli (a murine malaria parasite), a locus that surprisingly is both necessary and sufficient for invasion of epithelial cells by E. coli K12.

Initially, cosmid clones of segments of the P. yoeli genome were transduced into E. coli. The clones were screened for the ability to bind to and invade cultured COS-7 cells. In the screening process, primary validation of invasion or internalisation was based on gentamicin-killing; gentamicin permeates cells very poorly and can therefore be used to eliminate cell-surface bound bacteria.

On the basis of the screening method, several invasive clones (‘Inv’) were isolated and found to cross-hybridise.

Restriction mapping of the clones further showed that they overlapped with each other.

In order to confirm that the identified clones were indeed invasive, further invasion assays were carried out using E. colii K12 strains transformed with the cosmid clones.

The assays were carried out as above but the bacteria were tracked by indirect immunofluorescence using a primary polyclonal antibody specific to E. coli K12. Punctate fluorescence of invading bacteria was observed. No invasion was observed when the assay was carried out with control strains: untransformed E. coli or bacteria transformed with plasmid or cosmid vector.

To establish that the bacterial cells were internalised rather than extracellular, the invaded cells were counter-stained with TRITC-labelled phalloidin. Actin polymerisation could be observed at foci of cell entry and invading bacteria colocalised with the nucleation of polymerised actin filaments.

To provide further proof of cell entry, transmission electron microscopy was performed on the invaded cultured cells. Bacteria were found within membrane-bound vacuoles.

In order to investigate further the role of this locus in cell entry, one invasion-proficient clone, Invcos18, was subjected to transposon mutagenesis with Tn1000γδ (Morris, G. E et al, 1995).

97% of the transposon mutants were severely impaired for cell invasion, and were unable to complement E. coli in cell entry.

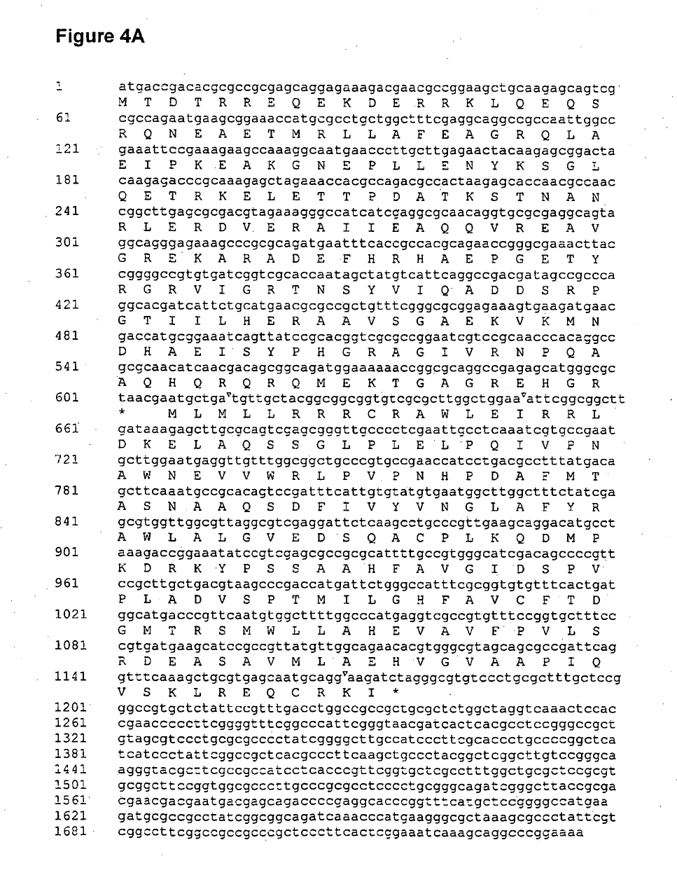

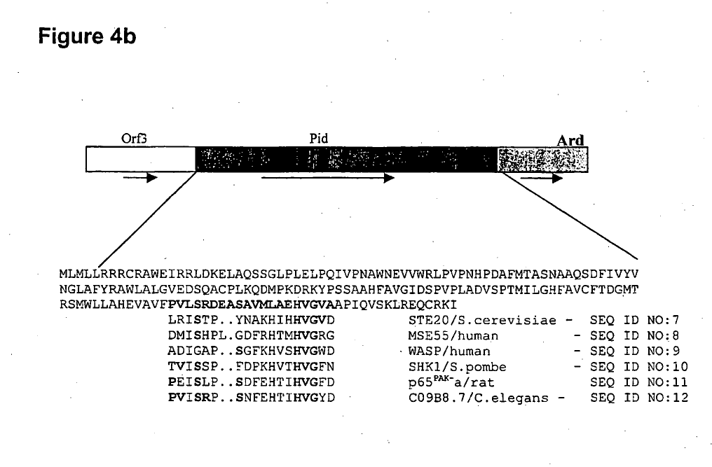

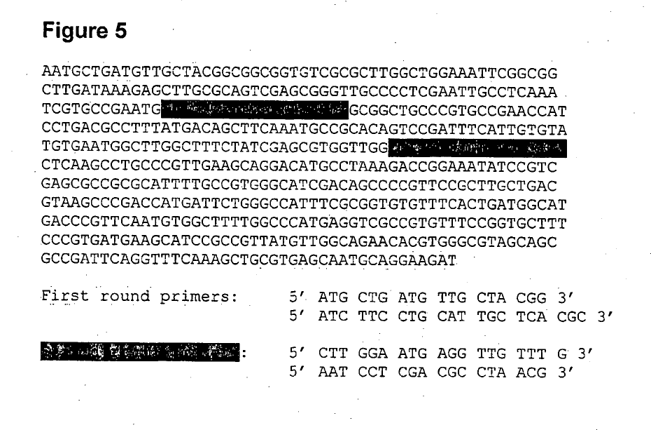

Sequence analysis of six non-complementing clones (with cfu≦1) showed transposon insertion at the same site in all six cases, suggesting that the inserted locus was indispensable for invasion. FIG. 4A shows the nucleotide sequence of the region, with the location of the transposon ends marked in sequence.

Transposon insertion sites were located which intercept a putative open reading frame of about 567 nucleotides. The sequence of the pid locus is designated SEQ ID no 2 and runs from nucleotide 607 in FIG. 4A to nucleotide 1172.

The predicted amino acid sequence of the pid translation product is presented in FIG. 4B and is designated SEQ ID No.1. This is also shown in FIG. 4A. The DNA ssequence upstream of Pid is designated SEQ ID NO:3 and encodes an amino acid sequence from ORF3, designated SEQ ID No.4. The sequence downstream of Pid is designated SEQ ID NO:5.

The BLAST programme was used to search the GenBank, EMBL, DDBJ and PDB databases for sequences homologous to the pid or Pid sequences.

The pid locus was found to be a pathogenicity island characterised by an unusually high GC content (55%) compared to parasite chromosomal DNA. DNA sequences contiguous with the invasion locus are more AT rich and show homology (about 38%) to the transmissible TraC/primase locus, which is required for conjugal transfer of the broad host range plasmids, IncN and IncW (Valentine, C. R. I. and C. I. Kado, 1989). Anatol, A. Belogurov et al, Antirestriction protein Ard (Type C) encoded by IncW plasmid pSa has a high similarity to the protein transport domain of Trac1 primase of promiscuous plasmid RP4. Journal of Molecular Biology, (2000), 296: 969-977.

Without wishing to be bound by theory, this suggests the presence of integrated cryptic plasmid DNA sequences in the parasite genome. It is possible that the pid locus has been acquired by horizontal transfer of a primordial gene derived from a pathogenic bacterium or bacteriophage.

In the homology searches, the Pid protein amino acid sequence was found to be identical to that of the Osa protein, encoded by the osa gene on the Shigella flexneri virulence plasmid pSa. The pid and osa nucleotide sequences are identical.

The Pid protein also shows homology to the product of the internalin B locus (InlB) in the inlAB operon of Listeria. monocytogenes, which has been shown to be indispensable for invasion (Gaillard, J. L. et al, 1991; Parida, S. K. et al, 1998).

The inventors have also identified a putative CDC42/Rac- interactive binding (CRIB) motif:
PVLSRDEASAVMLAEHVGVA

in the Pid protein sequence. This motif is designated SEQ ID No65. The motif is associated with small GTPase- effector proteins such as N-WASP, Ste20 and MSE55. An alignment of the sequences of Pid and other CRIB proteins is shown in FIG. 4B. The alignment was produced using the BLAST programme. The putative CRIB domain in pid does not align with the internalin B sequence. The homology of the latter and of the Rho GTPase-effector proteins was identified by a BLASTP search. The Accession numbers for pid homologues are as follows:

| | | |
|---|---|---|
| Internalin B. | Acc # | AF 121032 |
| Cdc42 effector protein (MSE55) | Acc # | XM_001058.1 |
| STE20 | Acc # | L04655 |
| WASP/human | Acc # | NM 000377 |
| p65^{PAK} -d/rat | Acc # | NM 017198 |

Apart from internalin, all the rest have CRIB domains.

WASP, Ste20 and MSE55 proteins are known to have an effect on actin cytoskeletal reorganisation (Burbelo, P. D. et al, 1999; Hall, A et al, 1998; Burbelo, P. D et al, 1998). In Salmonella typhimurium, for example, an interaction of N- WASP and SopE with the Rho GTPases CDC42 and Rac1, is required for invasion of non-phagocytic cells (Hardt, W. D et al, 1998; Chen, L. M. et al 1996; Susuki, T et al, 1998; Masol, P et al, 1998). Thus, Pid may be a guanine nucleotide exchange factor or a GTPase-activating protein.

Isolation and expression of Pid.

The pid gene may be isolated from a suitable parasite using standard cloning techniques, and the sequence information in SEQ ID No1 and No2. Fragments of the gene may be obtained using standard methods.

The pid gene or fragments of the gene may be expressed, and the protein products purified using conventional methods. For details of the above methods the reader is referred to Riggs, P., in Ausubel, F. M. et al (eds) Current Protocols in Molecular Biology John Wiley & Sons, Inc., New York, pp 16.6.1-16.6.14 (1994)

Vaccine Production.

Antibody-mediated Vaccine

Once an antigenic component has been obtained, a vaccine comprising the antigenic component may be produced by conventional means. The reader is directed towards the following references:
Jane L. Holley, Mike Elmore, Margaret Mauchline, Nigel Minton and Richard W. Titball.Cloning, expression and evaluation of a recombinant sub-unit vaccine against Clostridium botulinum type. F toxin [Abstract] [Full text] Vaccine 19 (2-3) 288-297 (2000)
Michael Theisen et al. Identification of a major B-cell epitope of the Plasmodium falciparum glutamate-rich protein (GLURP), targeted by human antibodies mediating parasite killing; Vaccine 19 (2-3) 204-212 (2000)
J H Tian, S Kumar, D C Kaslow, and L H Miller Comparison of protection induced by immunization with recombinant proteins from different regions of merozoite surface protein 1 of Plasmodium yoelii Infect. Immun. 1997 65: 3032-3036.
Bittle, J. L., Houghten, R. A., Alexander, H., Shinnick, T. M., Sutcliffe, J. G., Lerner, R. A., Rowlands, D. J. and Brown, F. 1982. Protection against foot-and-mouth disease by immunization with a chemically synthesized peptide predicted from the viral nucleotide sequence. Nature 298:30-33.
Harlow, E. & Lane, D. 1988. Antibodies: A laboratory manual, Cold Spring Harbour Laboratory Press, Cold Spring Harbour, New York.
Holder, A. A. and Freeman, R. R. 1981. Immunization against bloodstage-rodent malaria using purified parasite antigens, Nature, 294:361-364.
Hollingdale, M. R., Nardin, E. H., Tharavanij, S., Schwartz, A. L. and Nussenzweig, R. S. 1984. Inhibition of entry of sporozoites of Plasmodium falciparum and Plasmodium vivax sporozoites into cultured cells; an in vitro assay of protective antibodies. J. Immunol. 132:909-913.
Kaslow, D. C., Quakyi, I. A., Syin, C., Raum, M. G., Keister, D. B., Coligan, J. E., McCutchan, T. F. and Miller, L. H. 1988. A vaccine candidate from the sexual stage of human malaria that contains EGF-like domains. Nature 333:74-76.

The vaccine may be administered to a test subject and the antibodies raised in response to the vaccine tested for specific binding to Pid, both using standard methods. Suitable methods are described for example in the following references: J H Tian, S Kumar, D C Kaslow, and L H Miller Comparison of protection induced by immunization with recombinant proteins from different regions of merozoite surface protein 1 of Plasmodium yoelii .Infect. Immun. 1997 65: 3032-3036.
Harlow, E. & Lane, D. 1988. Antibodies: A laboratory manual, Cold Spring Harbour Laboratory Press, Cold Spring Harbour, New York.
Holder, A. A. and Freeman, R. R. 1981. Immunization against bloodstage-rodent malaria using purified parasite antigens, Nature, 294:361-364.
Hollingdale, M. R., Nardin, E. H., Tharavanij, S., Schwartz, A. L. and Nussenzweig, R. S. 1984. Inhibition of entry of sporozoites of Plasmodium falciparum and Plasmodium vivax sporozoites into cultured cells; an in vitro assay of protective antibodies. J. Immunol. 132:909-913.
Kaslow, D. C., Quakyi, I. A., Syin, C., Raum, M. G., Keister, D. B., Coligan, J. E., McCutchan, T. F. and Miller, L. H. 1988. A vaccine candidate from the sexual stage of human malaria that contains EGF-like domains. Nature 333:74-76.
Armando Reyna-Bello, Axel Cloeckaert, Nieves Vizcaííno, Mary I. Gonzatti, Pedro M. Aso, Gééraird Dubray, and Michel S. Zygmunt Evaluation of an Enzyme-Linked Immunosorbent Assay Using Recombinant Major Surface Protein 5 for Serological Diagnosis of Bovine Anapiasmosis in Venezuela Clin. Diagn. Lab. Immunol. 1998 5: 259-262.
M Theisen, J Vuust, A Gottschau, S Jepsen, and B Hogh Antigenicity and immunogenicity of recombinant glutamate- rich protein of Plasmodium falciparum expressed in Escherichia coli Clin. Diagn. Lab. Immunol. 1995 2: 30-34.

Similarly, the vaccine may be tested for effectiveness against disease such as those caused by apicomplexan parasites, using standard laboratory protocols. These may be, found, for example, in the following references: J H Tian, S Kumar, D C Kaslow, and L H Miller Comparison of protection induced by immunization with recombinant proteins from different regions of merozoite surface protein 1 of Plasmodium yoelii Infect. Immun. 1997 65: 3032-3036.
Harlow, E. & Lane, D. 1988. Antibodies: A laboratory manual, Cold Spring Harbour Laboratory Press, Cold Spring Harbour, New York.
Holder, A. A. and Freeman, R. R. 1981. Immunization against bloodstage-rodent malaria using purified parasite antigens, Nature, 294:361-364.
Hollingdale, M. R., Nardin, E.-H., Tharavanij, S., Schwartz, A. L. and Nussenzweig, R. S. 1984. Inhibition of entry of sporozoites of Plasmodium falciparum and Plasmodium vivax sporozoites into cultured cells; an in vitro assay of protective antibodies. J. Immunol. 132:909-913.
Kaslow, D. C., Quakyi, I. A., Syin, C., Raum, M. G., Keister, D. B., Coligan, J. E., McCutchan, T. F. and Miller, L. H. 1988. A vaccine candidate from the sexual stage of human malaria that contains EGF-like domains. Nature 333:74-76.
Armando Reyna-Bello, Axel Cloeckaert, Nieves Vizcaííno, Mary I. Gonzatti, Pedro M. Aso, Géérard Dubray, and Michel S. Zygmunt Evaluation of an Enzyme-Linked Immunosorbent Assay Using Recombinant Major Surface Protein 5 for Serological Diagnosis of Bovine Anaplasmosis in Venezuela Clin. Diagn. Lab. Immunol. 1998 5: 259-262.
M Theisen, J Vuust, A Gottschau, S Jepsen, and B Hogh Antigenicity and immunogenicity of recombinant glutamate- rich protein of Plasmodium falciparum expressed in Escherichia coli Clin. Diagn. Lab. Immunol. 1995 2: 30-34.
   Polynucleic Acid Vaccine

Once a suitable polynucleic acid has been identified, the polynucleic acid may be incorporated, in a vaccine using conventional methods. In this regard the reader is directed following documents: (Jones, T. R. et al, 1.999; Chatfield, S. N. et al, 1989;) Ricardo S. Corral and Patricia B. Petray CpG DNA as a Th1-promoting adjuvant in immunization against Trypanosoma cruzi Vaccine 19 (2-3) 234-242 (2000)
K. D. Song et. A DNA vaccine encoding a conserved Eimeria protein induces protective immunity against live Eimeria acervulina challenge Vaccine 19 (2-3) 243-252 (2000)
Rong Xiang, Holger N. Lode, Ta-Hsiang Chao, J. Michael Ruehlmann, Carrie S. Dolman, Fernando Rodriguez, J. Lindsay Whitton, Willem W. Overwijk, Nicholas P. Restifo, and Ralph
   A. Reisfeid An autologous oral DNA vaccine protects against murine melanoma PNAS 97: 5492-5497
Martha Sedegah, Trevor R. Jones, Manjit Kaur, Richard Hedstrom, Peter Hobart, John A. Tine, and Stephen L. Hoffman Boosting with recombinant vaccinia increases immunogenicity and protective efficacy of malaria DNA vaccine PNAS 95: 7648-7653.
Donald L. Lodmell, Nancy B. Ray and Larry C. Ewalt Gene gun particle-mediated vaccination with plasmid DNA confers protective immunity against rabies virus infection, Vaccine 16 (2-3) 115-118 (1998)
Cynthia L. Brazolot Millan, Risini Weeratna, Arthur M. Krieg, Claire-Anne Siegrist, and Heather L. Davis CpG DNA can induce strong Th1 humoral and cell-mediated immune responses against hepatitis B surface antigen in young mice PNAS 95: 15553-15558.
Wendy C. Brown, D. Mark Estes, Sue Ellen Chantler, Kimberly A. Kegerreis, and Carlos E. Suarez DNA and a CpG Oligonucleotide Derived from Babesia bovis Are Mitogenic for Bovine B Cells Infect. Immun. 1998 66: 5423-5432.
Zina Moldoveanu, Laurie Love-Homan, Wen Qiang Huang and Arthur M. Krieg. CpG DNA, a novel immune enhancer for systemic and mucosal immunization with influenza virus Vaccine, 16(11-12)1216-1224

The polynucleic acid vaccine may be administered to a subject and tested for therapeutic efficacy using standard protocols, such as those found in the following references:
J H Tian, S Kurrar, D C Kaslow, and T H Miller Comparison of protection induced by immunization with recombinant proteins from different regions of merozoite surface protein 1 of Plasmodium yoelii .Infect. Immun. 1997-65: 3032-3036.
Bittle, J. L., Houghten, R. A., Alexander, H., Shinnick, T. M., Sutcliffe, J. G., Lerner, R. A., Rowlands, D. J. and Brown, F. 1982. Protection against foot-and-mouth disease by immunization with a chemically synthesized peptide predicted from the viral nucleotide sequence. Nature 298:30-33.
Harlow, E. & Lane, D. 1988. Antibodies: A laboratory manual, Cold Spring Harbour Laboratory Press, Cold Spring Harbour, New York.
Holder, A. A. and Freeman, R. R. 1981. Immunization against bloodstage-rodent malaria using purified parasite antigens, Nature, 294:361-364.
Hollingdale, M. R., Nardin, E. H., Tharavanij, S., Schwartz, A. L. and Nussenzweig, R. S. 1984. Inhibition of entry of sporozoites of Plasmodium falciparum and Plasmodium vivax sporozoites into cultured cells; an in vitro assay of protective antibodies. J. Immunol. 132:909-913.
Kaslow, D. C., Quakyi, I. A., Syin, C., Raum, M. G., Keister, D. B., Coligan, J. E., McCutchan, T. F. and Miller, L. H. 1988. A vaccine candidate from the sexual stage of human malaria that contains EGF-like domains. Nature 333:74-76.
M Theisen, J Vuust, A Gottschau, S Jepsen, and B Hogh Antigenicity and immunogenicity of recombinant glutamate- rich protein of Plasmodium falciparum expressed in Escherichia coli Clin. Diagn. Lab. Immunol. 1995 2: 30-34.
   Design and production of therapeutic agent

Once a pid gene is isolated, and the Pid protein expressed and purified, a component for a therapeutic agent may be obtained by conventional methods.

The Pid protein may, for example, be crystallised and the 3-D structure determined, by methods such as those set out in Alex M. Aronov, Stephen Suresh, Frederick S. Buckner, Wesley C. Van Voorhis, Christophe L. M. J. Verlinde, Fred R. Opperdoes, Wim G. J. Hol, and Michael H. Gelb Structure- based design of submicromolar, biologically active inhibitors of trypanosomatid glyceraldehyde-3-phosphate dehydrogenase PNAS 96: 4273-4278.

This structure may be used as a template to design Pid mimics by combinatorial chemistry; the reader is directed towards the following documents: (Aronov, A. M. et al, 1999; ‘Combinatorial chemistry’ (1996) Methods in Enzymology vol 267 ed. John N Abelson; Academic Press Inc. New York;) Kirk McMillan, Marc Adler, Douglas S. Auld, John J. Baldwin, Eric Blasko, Leslie J. Browne, Daniel Chelsky, David Davey, Ronald E. Dolle, Keith A. Eagen, Shawn Erickson, Richard I. Feldman, Charles B. Glaser, Cornell Mallari, Michael M. Morrissey, Michael H. J. Ohlmeyer, Gonghua Pan, John F. Parkinson, Gary B. Phillips, Mark A. Polokoff, Nolan H. Sigal, Ronald Vergona, Marc Whitlow, Tish A. Young, and James J. Devlin Allosteric inhibitors of inducible nitric oxide synthase dimerization discovered via combinatorial chemistry PNAS 97: 1506-1511.
Dustin J. Maly, Ingrid C. Choong, and Jonathan A. Ellman Combinatorial target-guided ligand assembly: Identification of potent subtype-selective c-Src inhibitors PNAS 97: 2419-2424.
R A P Lutzke, N A Eppens, P A Weber, R A Houghten, and R H A Plasterk Identification of a Hexapeptide Inhibitor of the Human Immunodeficiency Virus Integrase Protein by Using a Combinatorial Chemical Library PNAS 92: 11456-11460
N F Sepetov, V Krchnak, M Stankova, S Wade, K S Lam, and M Lebl Library of Libraries: Approach to Synthetic Combinatorial Library Design and Screening of “Pharmacophore” Motifs PNAS 92: 5426-5430
J J Burbaum, M H J Ohlmeyer, J C Reader, I. Henderson, L W Dillard, G Li, T L Randle, N H Sigal, D Chelsky, and J J Baldwin A Paradigm for Drug Discovery Employing Encoded Combinatorial Libraries PNAS 92: 6027-6031
D. T. O'Hagan et al. Microparticles in MF59, a potent adjuvant combination for a recombinant protein vaccine against HIV-1 Vaccine 18(17):1793-1801 (2000)
Jing Huang and Stuart L. Schreiber A yeast genetic system for selecting small molecule inhibitors of protein-protein interactions in nanodroplets PNAS 94: 13396-13401.
Jay Parrish, Helen Metters, Lin Chen, and Ding Xue Demonstration of the in vivo interaction of key cell death regulators by structure-based design of second-site suppressors PNAS 97: 11916-11921

The mimics may be tested for competitive binding with Pid in a specific binding assay according to the methods set out in Harlow, E. & Lane, D. 1988. Antibodies: A laboratory manual, Cold Spring Harbour Laboratory Press, Cold Spring Harbour, New York.

The pharmacophores may be assessed by QSAR (quantitative structure-activity relationships); their toxicity may be tested in vivo and their efficacy as therapeutic agents assessed by the level of protection they confer from parasite infection. For suitable protocols, the reader is directed towards: Alex M. Aronov, Stephen Suresh, Frederick S. Buckner, Wesley C. Van Voorhis, Christophe L. M. J. Verlinde, Fred R. Opperdoes, Wim G. J. Hol, and Michael H. Gelb Structure-based design of submicromolar, biologically active inhibitors of trypanosomatid glyceraldehyde-3-phosphate dehydrogenase PNAS 96: 4273-4278.
NF Sepetov, V Krchnak, M Stankova, S Wade, K S Lam, and M Lebl Library of Libraries: Approach to Synthetic Combinatorial Library Design and Screening of “Pharmacophore” Motifs PNAS 92: 5426-5430
Bittle, J. L., Houghten, R. A., Alexander, H., Shinnick, T. M., Sutcliffe, J. G., Lerner, R. A., Rowlands, D. J. and Brown, F. 1982. Protection against foot-and-mouth disease by immunization with a chemically synthesized peptide predicted from the viral nucleotide sequence. Nature 298:30-33.
Harlow, E. & Lane, D. 1988. Antibodies: A laboratory manual, Cold Spring Harbour Laboratory Press, Cold Spring Harbour, New York.
Holder, A. A. and Freeman, R. R. 1981. Immunization against bloodstage-rodent malaria using purified parasite antigens, Nature, 294:361-364.
Hollingdale, M. R., Nardin, E. H., Tharavanij, S., Schwartz, A. L. and Nussenzweig, R. S. 1984. Inhibition of entry of sporozoites of Plasmodium falciparum and Plasmodium vivax sporozoites into cultured cells; an in vitro assay of protective antibodies. J. Immunol. 132:909-913.
Kaslow, D. C., Quakyi, I. A., Syin, C., Raum, M. G., Keister, D. B., Coligan, J. E., McCutchan, T. F. and Miller, L. H. 1988. A vaccine candidate from the sexual stage of human malaria that contains EGF-like domains. Nature 333:74-76.

The therapeutic agents may be delivered orally, in liposomes, by direct injection, or by controlled release from hydrophobic copolymers such as ethylene-vinyl acetate, cyclodextrins, polysialic acid or surfactant systems.

Suitable means are described in the following: (Ron, E. et al, 1993;); PNAS 90:4176-4180.

Alternatively, the receptor for Pid may be identified by protein-protein interaction trap assays, such as the yeast two-hybrid system, described in: (Chien, C-H. et al, 1991;); PNAS 88;9578-9582.

Inhibitors to this interaction may then be identified by further combinatorial chemistry, and duely optimised for inhibition by mutagenesis, using conventional methods. Suitable methods may be found in the following references: Kirk McMillan, Marc Adler, Douglas S. Auld, John J. Baldwin, Eric Blasko, Leslie J. Browne, Daniel Chelsky, David Davey, Ronald E. Dolle, Keith A. Eagen, Shawn Erickson, Richard I. Feldman, Charles B. Glaser, Cornell Mallari, Michael M. Morrissey, Michael H. J. Ohlmeyer, Gonghua Pan, John F. Parkinson, Gary B. Phillips, Mark A. Polokoff, Nolan H. Sigal, Ronald Vergona, Marc Whitlow, Tish A. Young, and James J. Devlin Allosteric inhibitors of inducible nitric oxide synthase dimerization discovered via combinatorial chemistry PNAS 97: 1506-1511
Dustin J. Maly, Ingrid C. Choong, and Jonathan A. Ellman Combinatorial target-guided ligand assembly: identification of potent subtype-selective c-Src inhibitors PNAS 97: 2419-2424.
R A P Lutzke, N A Eppens, P A Weber, R A Houghten, and R H A Plasterk Identification of a Hexapeptide Inhibitor of the Human Immunodeficiency Virus Integrase Protein by Using a Combinatorial Chemical Library PNAS 92: 11456-11460.
J J Burbaum, M H J Ohlmeyer, J C Reader, I Henderson, L W Dillard, G Li, T L Randle, N H Sigal, D Chelsky, and J J Baldwin A Paradigm for Drug Discovery Employing Encoded Combinatorial Libraries PNAS 92: 6027-6031
D. T. O'Hagan et al. Microparticles in MF59, a potent adjuvant combination for a recombinant protein vaccine against HIV-1 Vaccine 18(17):1793-1801 (2000)
Jing Huang and Stuart L. Schreiber A yeast genetic system for selecting small molecule inhibitors of protein-protein interactions in nanodroplets PNAS 94: 13396-13401.
Jay Parrish, Helen Metters, Lin Chen, and Ding Xue Demonstration of the in vivo interaction of key cell death regulators by structure-based design of second-site suppressors PNAS 97: 11916-11921.

The inhibitors may then be incorporated into therapeutic agents for receptor occlusion, and tested for therapeutic efficacy using standard methods.
Bittle, J. L., Houghten, R. A., Alexander, H., Shinnick, T. M., Sutcliffe, J. G., Lerner, R. A., Rowlands, D. J. and Brown, F. 1982. Protection against foot-and-mouth disease by immunization with a chemically synthesized peptide predicted from the viral nucleotide sequence. Nature 298:30-33.
Holder, A. A. and Freeman, R. R. 1981. Immunization against bloodstage-rodent malaria using purified parasite antigens, Nature, 294:361-364.
Kaslow, D. C., Quakyi, I. A., Syin, C., Raum, M. G., Keister, D. B., Coligan, J. E., McCutchan, T. F. and Miller, L. H. 1988. A vaccine candidate from the sexual stage of human malaria that contains EGF-like domains. Nature 333:74-76.
   Diagnostic agent production and method of diagnosis Diagnostic agent comprising antibody

Following expression of the Pid protein or a fragment thereof, methods for raising antibodies to the protein are well known in the art. Suitable methods may be found in the following references: Bittle, J. L., Houghten, R. A., Alexander, H., Shinnick, T. M., Sutcliffe, J. G., Lerner, R. A., Rowlands, D. J. and Brown, F. 1982. Protection against foot-and-mouth disease by immunization with a chemically synthesized peptide predicted from the viral nucleotide sequence. Nature 298:30-33.
Harlow, E. & Lane, D. 1988. Antibodies: A laboratory manual, Cold Spring Harbour Laboratory Press, Cold Spring Harbour, New York.
Holder, A. A. and Freeman, R. R. 1981. Immunization against bloodstage-rodent malaria using purified parasite antigens, Nature, 294:361-364.
Hollingdale, M. R., Nardin, E. H., Tharavanij, S., Schwartz, A. L. and Nussenzweig, R. S. 1984. Inhibition of entry of sporozoites of Plasmodium falciparum and Plasmodium vivax sporozoites into cultured cells; an in vitro assay of protective antibodies. J. Immunol. 132:909-913.
Kaslow, D. C., Quakyi, I. A., Syin, C., Raum, M. G., Keister, D. B., Coligan, J. E., McCutchan, T. F. and Miller, L. H. 1988. A vaccine candidate from the sexual stage of human malaria that contains EGF-like domains. Nature 333:74-76.

These antibodies may be incorporated into a diagnostic agent and used in diagnosis according to conventional methods. The reader is directed towards the following publications: Shuli Li, Gina Galvan, Fausto G. Araujo, Yasuhiro Suzuki, Jack S. Remington, and Stephen Parmley. Serodiagnosis of Recently Acquired Toxoplasma gondii Infection Using an Enzyme-Linked Immunosorbent Assay with a Combination of Recombinant Antigens Clin. Diagn. Lab. Immunol. 2000 7: 781-787.
Kerstin Hubert, Abel Andriantsimahavandy, Alain Michault, Matthias Frosch, and Fritz A. Müühlschlegel Serological Diagnosis of Human Cysticercosis by Use of Recombinant Antigens from Taenia solium Cysticerci Clin. Diagn. Lab. Immunol. 1999 6: 479-482.
Byoung-Kuk Na and Chul-Yong Song Use of Monoclonal Antibody in Diagnosis of Candidiasis Caused by Candida albicans: Detection of Circulating Aspartyl Proteinase Antigen Clin. Diagn. Lab. Immunol. 1999 6: 924-929.
Felix Grimm, Friedrich E. Maly, Jian Lüü, and Roberto Llano Analysis of Specific Immunoglobulin G Subclass Antibodies for Serological Diagnosis of Echinococcosis by a Standard Enzyme-Linked Immunosorbent Assay Clin. Diagn. Lab. Immunol. 1998 5: 613-616.
Armando Reyna-Bello, Axel Cloeckaert, Nieves Vizcaííno, Mary I. Gonzatti, Pedro M. Aso, Géérard Dubray, and Michel S. Zygmunt Evaluation of an Enzyme-Linked Immunosorbent Assay Using Recombinant Major Surface Protein 5 for Serological Diagnosis of Bovine Anaplasmosis in Venezuela Clan. Diagn. Lab. Immunol. 1998 5: 259-262.
Franççois Simondon, Isabelle Iteman, Marie Pierre Preziosi, Abdoulaye Yam, and Nicole Guiso Evaluation of an Immunoglobulin G Enzyme-Linked Immunosorbent Assay for Pertussis Toxin and Filamentous Hemagglutinin in Diagnosis of Pertussis in Senegal Clin. Diagn. Lab. Immunol. 1998 5: 130-134.
I Bjerkas, M C Jenkins, and J P Dubey Identification and characterization of Neospora caninum tachyzoite antigens useful for diagnosis of neosporosis Clin. Diagn. Lab. Immunol. 1994 1: 214-221.
M Theisen, J Vuust, A Gottschau, S Jepsen, and B Hogh Antigenicity and immunogenicity of recombinant glutamate-rich protein of Plasmodium falciparum expressed in Escherichia coli Clin. Diagn. Lab. Immunol. 1995 2: 30-34.
Laurens A. H. van Pinxteren, Pernille Ravn, Else Marie Agger, John Pollock, and Peter Andersen Diagnosis of Tuberculosis Based on the Two Specific Antigens ESAT-6 and CFP10 Clin. Diagn. Lab. Immunol. 2000 7: 155-160.

E. E. Zijlstra, N. S. Daifalla, P. A. Kager, E. A. G. Khalil, A. M. El-Hassan, S. G. Reed, and H. W. Ghalib rK39 Enzyme-Linked Immunosorbent Assay for Diagnosis of Leishmania donovani Infection Clin. Diagn. Lab. Immunol. 1998 5: 717-720.

Diagnostic Agent Comprising Antigen

An effective antigenic component may be obtained as described above. The antigenic component may then be incorporated in a diagnostic agent and used in diagnosis according to standard methods.

Suitable methods are described in the following documents: (Mills, C. D. et al, 1999;); Bull WHO 77:553-559; Felix Grimm, Friedrich E. Maly, Jian Liu, and Roberto Llano Analysis of Specific Immunoglobulin G Subclass Antibodies for Serological Diagnosis of Echinococcosis by a Standard Enzyme-Linked Immunosorbent Assay Clin. Diagn. Lab. Immunol. 1998 5: 613-616.

Armando Reyna-Bello, Axel Cloeckaert, Nieves Vizcaííno, Mary I. Gonzatti, Pedro M. Aso, Géérard Dubray, and Michel S. Zygmunt Evaluation of an Enzyme-Linked Immunosorbent Assay Using Recombinant Major Surface Protein 0.5 for Serological Diagnosis of Bovine Anaplasmosis in Venezuela Clin. Diagn. Lab. Immunol. 1998 5: 259-262.

Franççois Simondon, Isabelle Iteman, Marie Pierre Preziosi, Abdoulaye Yam, and Nicole Guiso Evaluation of an Immunoglobulin G Enzyme-Linked Immunosorbent Assay for Pertussis Toxin and Filamentous Hemagglutinin in Diagnosis of Pertussis in Senegal Clin. Diagn. Lab. Immunol. 1998.5: 130-134.
Bjerkas, MC Jenkins, and JP Dubey Identification and characterization of Neospora caninum tachyzoite antigens useful for diagnosis of neosporosis lin. Diagn. Lab. Immunol. 1994 1: 214-221.
M Theisen, J Vuust, A Gottschau, S Jepsen, and B Hogh Antigenicity and immunogenicity of recombinant glutamate-rich protein of Plasmodium falciparum expressed in Escherichia coli Clin. Diagn. Lab. Immunol. 1995 2: 30-34. Laurens A. H. van Pinxteren, Pernille Ravn, Else Marie Agger, John Pollock, and Peter Andersen Diagnosis of Tuberculosis Based on the Two Specific Antigens ESAT-6 and CFP10 Clin. Diagn. Lab. Immunol. 2000 7: 155-160.
E. E. Zijlstra, N. S. Daifalla, P. A. Kager, E. A. G. Khalil, A. M. El-Hassan, S. G. Reed, and H. W. Ghalib rK39 Enzyme-Linked Immunosorbent Assay for Diagnosis of Leishmania donovani. Infection Clin. Diagn. Lab. Immunol. 1998 5: 717-720.
   Materials and Methods

1. Construction of Cosmid Library of P. yoelii Genomic DNA.

High molecular weight genomic DNA was isolated from an asynchronous culture of P. yoelii and partially cleaved with Sau3A to yield fragments of 30-50 kb. These were dephosphorylated with calf intestinal alkaline phosphatase and ligated into the cosmid vector SuperCos1 (Stratagene) previously digested with BamH1. An aliquot of the ligation mixture was packaged with Gigapack III XL packaging extract (Stratagene) and transduced into XL1-Blue MR (Stratagene) Recombinant cosmids were selected on ampicillin, pooled and amplified once.

2. Primary Screening of Cosmid Clones for Invasion of Cultured COS-7 Cells.

For invasion assays, a logarithmic phase culture of E. coli harbouring the cosmids were seeded onto COS-7 cells at a multiplicity of infection of 10, and invasion assays performed essentially as described. After 5 h of incubation the cells were washed extensively and fresh medium containing 250 μg/ml gentamicin was added to kill non-invading bacteria. After an overnight incubation, the cells were washed 10× with PBS. Invading bacteria were released by gentle lysis of the COS-7 cells with PBS/0.05% saponin, and scored by ampicillin selection on LB-plates.

3. Immunofluorescence and Electron Microscopy Studies.

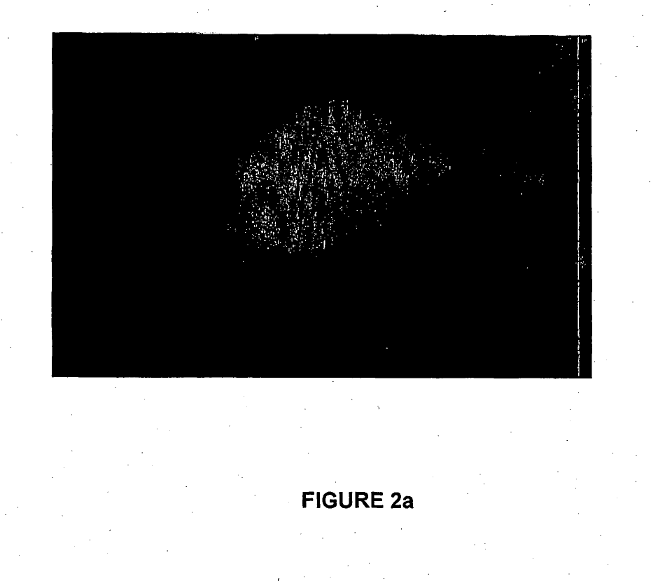

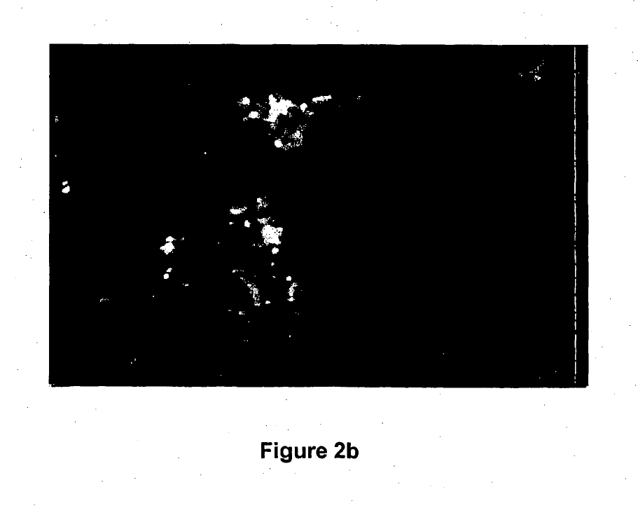

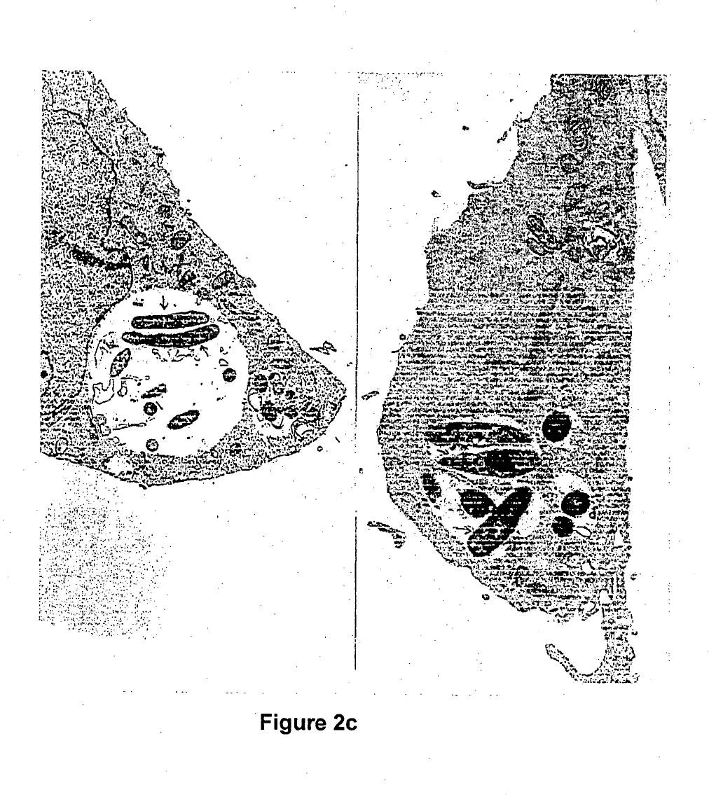

Invasion assays were performed as above. The cells were then washed extensively, and fixed with 3.7% paraformaldehyde. The cells were permeabilised at room temperature for 30 min with PBS/0.05% saponin, incubated with 5% non-fat dry milk in PBS. After 1 h at room temperature, the cells were washed 3× witch PBS and incubated with a rabbit polyclonal antibody to the E. coli K-12 strain C600 (Dako Ltd). This antibody cross-reacts with other K-12 strains. Following incubation for 2 h, the cells were washed with PBS and incubated with rabbit FITC-labelled IgG (Sigma) for 1 h, washed extensively with PBS and mounted for fluorescence microscopy on a Zeiss microscope. The results are illustrated in FIG. 2A (negative control) and FIG. 2B infections with E. coli containing invasion cosmid clone.

Cells may also be counter stained with TRITC-phalloidin to determine bacteria and actin polymer colocalisation.

In order to obtain electron micrographs of COS-7 cells invaded by transformed E. coli, invasion assays were carried out as above, and the COS-7 cells then washed extensively with PBS before fixing with 4% glutaraldehyde overnight at 4° C. The fixed cells were sectioned, stained with osmium tetroxide and viewed with a transmission electron microscope. The results are shown in FIG. 2C.

4. Transposon Mutagenesis of Identified Invasive Clones, and Sequence Analysis.

Transposon mutagenesis was performed using the Tn1000γδ, essentially as described, with minor modifications as follows.

Two invasive clones Invcos11 and Invcos18 were transformed into competent MH1638 donor cells. Single colonies were grown to log phase in LB/100 μg per ml ampicillin/50 μg per ml methicillin. Recipient cells, HB101 were grown in LB. 2 ml of donor culture were spun and resuspended with 15 ml of LB in Falcon tubes. After centrifuging for 10 min at 4500 rpm, the cell pellet was resuspended with 1 ml of recipient HB101 culture. This mating mixture was spread on LB agar plates (without antibiotics) and incubated for 2 h at 37° C.

The cells were washed with 15 ml LB broth and centrifuged as above. After a second spin, the pellet was resuspended in 1 ml LB broth. 100 μl of this was plated on LB plates supplemented with 100 μg/ml ampicillin, 501 g/ml methicillin and 1001 g/ml streptomycin. After an overnight incubation at 37° C., 50 isolated colonies were picked, grown in LB/ampicillin/methicillin/streptomycin (LB/amp/meth/strep), and used in invasion assays as described above.

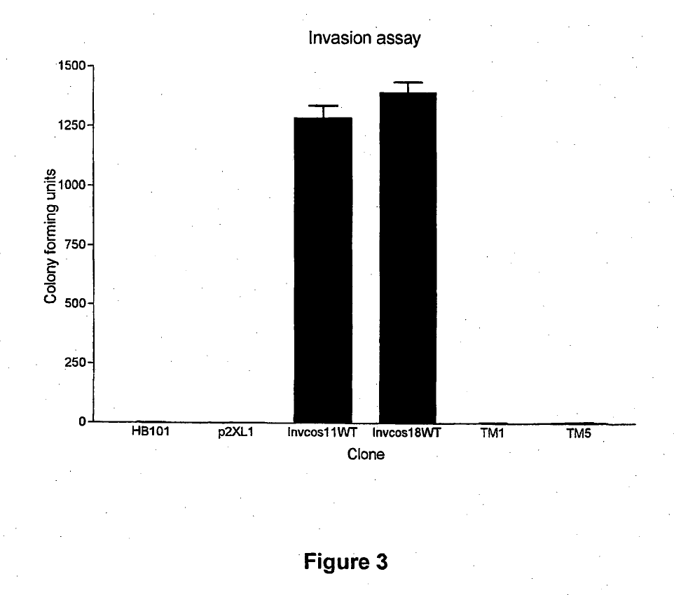

Invading colonies were scored by plating saponin-lysed COS-7 cells on LB/amp/meth/strep. FIG. 3a shows plates obtained in plate scoring after invasion assays using a wild type and a Tn1000γδ-inserted clone.

FIG. 3b provides a bar chart displaying the results of plate scoring following invasion assays using Tn1000γδ-inserted clones TM1 and TM5. Wild type Invcos11 and Invcos18 were used as positive controls for invasion, while HB101 or XL1 Blue transformed with pMAL-p2 (New England Biolabs) grown on LB agar or LB/amp agar acted as negative controls.

Non-complementing clones identified from this assay were purified and sequenced using the following primers:

| | | |
|---|---|---|
| γ | CCTGAAAAGGGACCTTTGTATACTG | (SEQ ID No 13) |
| δ | AGGGGAACTGAGAGCTCTA | (SEQ ID No 14) |

The sequence of the inserted region, shown in FIG. 4A, was obtained by contig assembly of the sequences from the transposon mutants.
Antigenic Responses to Pid
Methods

1. Sub-Cloning of pid and expression of Pid-Myc fusion protein. pid was prepared as a PCR amplimer from invcos18 with HindIII and Bg1 restriction sites at the 5′ and 3′ ends respectively. The amplimer was sub-cloned into the expression vector pROlar A122(Clontech) which includes a Myc epitope tag. Colonies were maintained on LB/kanamycin plates at 37° C. A 50 μl aliquot of an over night broth culture was used to inoculate a 5 ml broth and this was incubated to 0.4-0.6 OD₆₀₀, at this point the culture was induced with 5 μl 100 mM IPTG and 53 μl 15% arabinose. The culture was incubated for 3 hours at 37° C.

2. Pid preparation. Concentration of Pid was enhanced using c-Myc monoclonal antibody-agarose beads (Clontech). Following induction cells were washed in ice-cold PBS, the pellet was then re-suspended in lysis buffer and frozen at −70° C. The sample was then thawed on ice and 2041 c-Myc Mab was added, vortexed briefly and mixed on a rotating platform for 40 min at 4° C. The preparation was then washed 3 times in ice cold PBS, with microcentrifugation at 1200 g for 1 min.

3. Patient sera. Serum was collected from 8 patients admitted to the Royal Free Hospital with malaria. Plasmodium falciparum infection and degree of parasitaemia confirmed. Country of origin was noted.

4. Western blot analysis. Multiple lanes of Pid preparation were separated by 12% SDS-PAGE prior to electrophoretic transfer to Hybond-C (AmershamPharmacia), transfer was for 2 h at 10V (Novablot, AmershamPharmacia). The resultant blots were probed with each patient serum at a dilution of 1/100 aid visualised using the ECL Western Blotting reagents (AmershamPharmacia)

Results

Five out of eight patient sera tested recognised a protein band that co-migrated with the Pid-Myc fusion product. These sera did not recognise a band of this molecular weight when cells transformed with pROlar containing an unrelated non-Plasmodium sequence were tested. The country of origin of each patient and levels of parasiteamia are shown in Table 1.

### CONCLUSION

The preparation of E. coli cells with induced expression of Pid did show the presence of an antigenic moiety of the predicted molecular weight in 5/8 sera tested. The patients all had marked parasitaemia and were from sub-saharan Africa. This data indicates that patients exposed to Plasmodium falciparum do raise an immune response to Pid and therefore this may form the basis of a serological assay for the detection of this infection.

TABLE 1

| | Parasitaemia | Country | Positive on |
|---|---|---|---|
| Patient | % | of origin | Western blot |
| 1 | 3.8 | Ghana | + |
| 2 | <1.0 | Ghana | ++ |
| 3 | <1.0 | Ghana | − |
| 4 | 1.2 | Ghana | −/+ |
| 5 | 4.5 | Tanzania | ++ |
| 6 | <1.0 | Nigeria | − |
| 7 | <1.0 | Ghana | + |
| 8 | <1.0 | Nigeria | ++ |

PCR Amplification of Pid from Patient Sera.
Method

1. PCR amplification of Did: Using genomic DNA from falciparum T9-96 a nested PCR protocol was using the following primers; first round 5′ ATG CTG ATG TTG CTA CGG 3′, pfpid2, 5′ATC TTC CTG CAT TGC TCA CGC 3′ and Second round primers, pfpid3 5′ CTT GGA ATG AGG TTG TTT G 3′ and pfpid4 5′ AAT CCT CGA CGC CTA ACG 3′ (FIG. 1). The optimised reaction mixture for the first round PCR was, 1×NH₄Cl₂ stock buffer (Biolline, UK), 5 mM MgCl₂, 1 μM pfpid1, 1 μM pfpid2, 100 μM dNT2, 2.5 IU Taq polymerase (Bioline, UK). Cycling conditions were 95° C. for 3 min, 45 cycles of 92° C. 30 sec, 50° C. 30 sec, 72° C. 30 sec followed by a cycle of 72° C. 5 min. For the nested PCR 2 μl of the amplimer preparation from the first round PCR was added to the reaction mix; 1× NH₄Cl₂ stock buffer (Bioline, UK), 5 mM MgCl₂, 1 μM pfpid3, 1 μM pfpid4, 200 μM dNTP, 2.5 IU Taq polymerase (Bioline, UK). Cycling conditions were 95° C. for 3 min, 45 cycles of 92° C. 30 sec, 55° C. 30 sec. 72° C. 30 sec followed by a cycle of 72° C. 5 min.

2. Patient samples. Whole blood was collected from 8 patients admitted to the Royal Free Hospital with malaria. Fresh whole blood was centrifuged at 3,000 g for 5 mins. The serum was removed and the red cells stored at −20 CC. Plasmodium falciparum infection and degree of parasitaemia confirmed. Country of origin was noted.

3. PCR amplification of pid from patient samples: DNA was extracted from the red cell pellet, according to the manufacturers instructions using the Wizard® Genomic DNA Purification Kit (Promega, UK). 5 μl of the resultant genomic DNA was then amplified according to the optimised protocol in section 1. PCR amplimers were visualised on a 2% agarose gel containing ethidium bromide.

4. Sequence analysis of PCR amplimers from patient samples: PCR amplimers of the predicted molecular weight were purified using the Wizard® PCR Purification Kit (Promega, UK). Amplimers were then submitted for sequencing to Cambridge Biosciences (now Cytomyx, UK).

Results

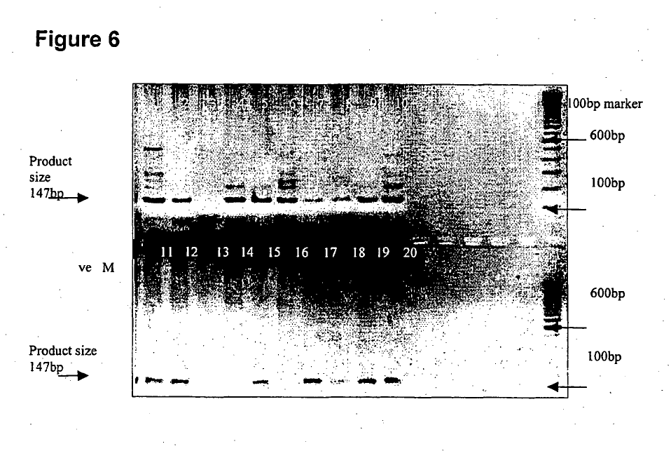

FIG. 6 shows the results of PCR amplification of pid from patient samples; 17/20 samples gave positive results, this represents positive results from all patients tested, 3 patients had one negative result. Patient details and PCR results are summarised in Table 2.

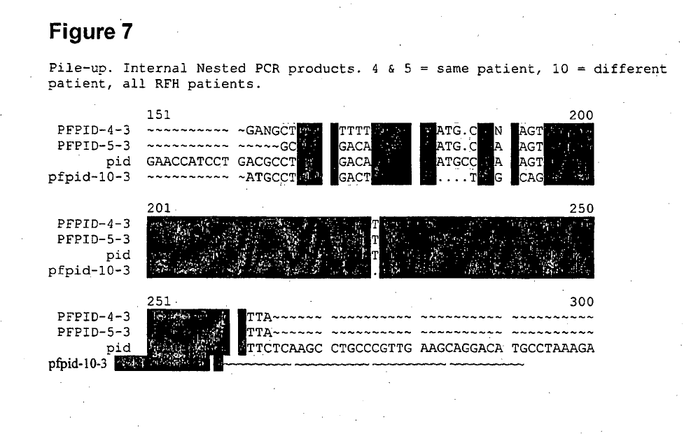

Sequencing was performed using amplimers from the PCR reactions giving the strongest results, samples 4, 5 and 10. Analysis of the similarity of these sequences to pid was performed using PILEUP (GCG, MRC-HGMP UK), samples 4 and 5 came from the same patient, all 3 samples show a high degree of similarity to pid (FIG. 7).

### CONCLUSION

This data provides evidence that pid can be amplified from patients with confirmed Plasmodium falciparum infection and thus may be a suitable target for a diagnostic test. The PCR amplimers investigated for similarity to pid showed a high degree of homology, confirming their identity as pid.

TABLE 2

| | Sample | | Parasitaemia | |
|---|---|---|---|---|
| Patient | Number | Country of origin | % | pid PCR |
| A | 1 | Ghana | 3.5 | + |
| | 2 | | 3.8 | + |
| | 3 | | <1.0 | − |
| B | 4 | Ghana | <1.0 | + |
| | 5 | | 0 | + |
| | 14 | | 0 | − |
| C | 6 | Ghana | <1.0 | + |
| | 7 | | <1.0 | + |
| | 8 | | 0 | + |
| | 9 | | 0 | + |
| D | 10 | Tanzania | 1.2 | + |
| | 11 | | 4.5 | + |
| | 12 | | <1.0 | + |
| | 13 | | 0 | − |
| E | 15 | Nigeria | <1.0 | + |
| | 16 | | <1.0 | + |
| F | 17 | ? | <1.0 | + |
| | 18 | | <1.0 | + |
| G | 19 | ? | <1.0 | + |
| | 20 | | 1.5 | + |

pid mediated invasion of human red blood cells
Methods

1. Bacterial cell lines: E. coli K12 strain XL1-Blue MR transformed with either invcos18, pROLAR-pid or native pROLAR were used. These constructs are described above.

2. Human red blood cells (rbc): 10 ml freshly drawn blood was transferred to a tube containing lithium heparin. The blood donor had no history of travel to an area endemic for malaria in any form and had no known exposure to malaria. The rbc were separated by centrifugation at 3,000 g for 5 min and washed 3 times in DMEM/10% foetal bovine serum (FBS) by microcentrifugation at 5000 g.

3. Invasion assay: Transformed E. coli were incubated with red blood cells at a ratio of 10:1 (85×10⁵ bacteria: 8.5×10⁵ rbcs) for 3 h at 37° C. in 5% CO₂. The cells were then washed 3 times in DMEM/10% FBS by microcentrifugation at 5000 g. The washed cells were resuspended in DMEM/FBS, 200 μg/ml gentamicin was added and the preparation was incubated overnight at 37° C. in 5% CO₂. Following incubation the cells were washed 3 times in DMEM/10% FBS by microcentrifugation at 5000 g and the rbc were lysed by resuspension in sterile distilled water. 50 μl of the lysates was spread on LB agar plates containing kanamycin, lysates were plated in duplicate.

Plates were read after 24 h and the number of colonies counted by 2 independent observers.

Results

Colony counts are shown in Table 3.

### CONCLUSION

The data presented in Table 3 indicate that invasion of human red blood cells by E. coli can be mediated by the presence of pid alone, however, invasion is enhanced by the presence of the complete cosmid, suggesting the presence of further genes that contribute to the invasive process.

TABLE 1
pid mediated invasion of fresh human red blood cells

| | Duplicate colony counts | |
|---|---|---|
| Construct | Observer 1 | Observer 2 |
| pROLAR | 46/48 | 51/47 |
| pROLAR-pid | 88/90 | 97/99 |
| Invcos18 | 780/881 | 847/743 |

INTERACTION BETWEEN PID AND THE RHO GTPASES

cDNAs encoding human Cdc42, Rac1 and RhoA were amplified by PCR with the following set of primers:

Cdc42: sense (cag gaa ttc cag aca att aag tgt gtt g); antisense (cag gtc gac tta gaa tat aca gca ctt cc). Rac1: sense (cag gaa ttc cag gcc atc aag tgt gtg); antisense (cag gtc gac cta caa cag gca ttt tct c). RhoA: sense (cag gaa ttc gct gcc atc cgg aag aaa ctg); antisense (cag cgt cga ctc aca aga caa ggc aac c). All three GTPases were digested with EcoRI and SalI and ligated into the same sites in the activation domain vector pAD-GAL4 (Stratagene). Pid was also amplified (sense: cag gga att cat gct gat gtt gct ac); antisense (cag cgt cga cct aga tct tcc tgc), digested with the above enzymes and ligated into the binding-domain vector pBD-GAL4 (Stratagene). All 4 legations were used to transform competent DH5α cells and selected on LB agar/ampicillin plates. Transformants were screened for inserts, and these inserts were restriction-mapped to determine whether they were n the right orientation, with respect to the GAL4 fusion domains. The new plasmids were designated AD-Cdc42, AD-Rac1, AD-RhoA and BD-Pid for the respective proteins.

To determine interaction, the AD-GTPase constructs were each cotransformed with BD-Pid into competent yeast cells YRG-2 (Stratagene). These were then plated on synthetic dextrose agar plates (SD/-His-Leu-Trp). This medium lacks the amino acids leucine, (which selects for activation domain-GTPase constructs), tryptophan (which selects for binding domain-Pid) and histidine, which selects for the HIS reporter gene. After 3-7 days incubation at 30° C., colonies were isolated and grown in SD liquid medium for 3 days at 30° C. in a shaking incubator.

Observation: Interaction between Pid and the RhoA GTPases was scored based on both amino acid prototrophy and the expression of the HIS reporter gene. However, only Cdc42 appeared to be definitely interacting with Pi-d.

Subcellular Localization of PID

Using a reporter protein, the green or red fluorescent protein, it is often possible to track the location of a protein using cells under culture conditions. To do this, Pid was PCR-amplified With the primers: cag gga att cat gct gat gtt gct ac (sense) and antisense (cat gct cga gat ctt cct gca ttg ctc ac) and digested with EcoRI and XhoI. It was then ligated into the EcoRI/SalI sites of pDsRed-N1 (Clontech), at the N-terminus and in-frame with the red fluorescent protein. Transformants were derived from DH5a cells, which were selected on kanamycin LB agar plates. Plasmid DNA was purified and the presence and orientation of Pid insert were determined by restriction mapping. The plasmid was designated pPid-DsRed.

HeLa cells (˜10⁴ cells) were seeded on Permanox chamber slides and grown in Dulbeccos Modified Eagles Medium (DMEM) supplemented with 10% foetal bovine serum, antimycotics and antibiotics, at 37° C. under 5% CO₂. At about 80% confluence the cells were rinsed and then incubated with fresh medium 1-2 hr before transfection. pPid-DsRed (5 μg) was diluted in OptiMEM medium to a final volume of 100 μl. 10 μl liposomes (DOSPER, Roche) was also diluted to the same volume. Both plasmid DNA and liposome dilutions were mixed and incubated at room temperature for 30 min to allow complex formation. The mixture was then transferred with a pipette onto the HeLa cells and incubation continued for 6-12 hrs. Fresh DMEM was then added and the cells incubated for another 30 hrs. After this time, the cells were washed 2× with PBS, and fixed with 3.7% paraformaldehyde for 30 mins at room temperature. They were then mounted in Vectashield Mounting Medium with DAPI for nuclear counter-staining (Vector Laboratories) and viewed under a Nikon Eclipse E800 fluorescent microscope. Images were acquired from 2 micron sections with the Bio-Rad Radiance 2100 confocal microscope.

Observation: In HeLa cells transfected with pDsRed-N1, fluorescence was observed throughout the entire cell, both in the cytosol and nuclear lumen. In contrast, in cells transfected with pPid-DsRed, fluorescence appeared to be confined within granules (possibly secretory) or peripheral vesicles that were in juxtaposition to the plasma membrane.

ACTIN DEPOLYMERIZATION IMPEDES CELL INVASION BY PID-TRANSFORMED BACTERIA

In earlier experiments it was observed by double staining with FITC-labelled antibodies and TRITC-phalloidin, that bacterial cells transformed with the invasion cosmid induced actin nucleation/polymerisation at foci of cell entry. This suggested the recruitment of cytoskeletal components in the invasion process. Taking this a step further, it was considered whether actin depolymerisation had any effect on the invasion process. To address this, 10⁴ HeLa cells were seeded onto Thermanox cover slips and cultured in DMEM as above. Before invasion assays, the HeLa cells were washed 2× with PBS and then incubated with fresh medium lacking antibiotics. For inhibition, cytochalasin B was added to the cells at a concentration of 0.5-1 μg/ml, 2-5 mins before invasion assays were initiated. Bacterial cells containing pA15-Pid (Pid subcloned into pROLar.A122), pGEX-Pid (Pid subcloned into pGEX-5×1), Incos18 (wild-type invasion cosmid), In18-M1 (mutant cosmid) or untransformed HB101 were then added to the HeLa cell culture at an m.o.i of 10. To control for invasion efficiency, cytochalasin B was omitted in parallel assays with the transformed bacteria. After 3-5 hr, the cells were washed 5× with PBS and then incubated with DMEM supplemented with −200 μg/ml gentamicin and left overnight in the incubator. The cells were then washed with PBS (5×), fixed with 3.7% paraformaldehyde and permeabilized with PBS/0.05% saponin for 30 min at room temperature. They were incubated for 1 hr at room temperature with. PBS/5% non-fat dry milk and then with anti-E. coli antibody diluted in PBS/5% non-fat dry milk. They were rinsed 2× with PBS and incubated again for 1 hr with anti-rabbit IgG-FITC conjugate alone or with TRITC-phalloidin. The cells were Lhen washed extensively with PBS, and mounted for fluorescence microscopy.

Observation: HeLa cells treated with cytochalasin B were refractory to invasion by pA15-Pid or pGEX-Pid-transformed bacteria. Incos18-transformed cells showed a localization of the bacterial cells at the periphery of the cytochalasin B-treated HeLa cells if they were present. This showed the uncoupling of bacterial cell attachment to the HeLa cells from the invasion step. HeLa cells untreated with cytochalasin B were efficiently invaded by Incos18, pGEX-Pid and pA15-Pid but not by In18TM1 or HB101.

Corresponding Pid Sequence in Cryptosporidium

Cryptosporidium parvum oocysts were obtained from Moredun Scientific Ltd, Scotland. Approximately 100,000 oocysts were centrifuged and respuspended in 200 μl of 50 mM Tris-HCl pH 8.0, 5 mM EDTA, 50 mM NaCl, 0.5% Sarkosyl, 100 ug/ml proteinase K. The cells were incubated for 2 hrs at 60° C. in a PCR machine. The lysate was then extracted 2× with phenol-chloroform-isoamyl alcohol. Genomic DNA was precipitated with 0.1 volume of 3M sodium acetate pH 5.2 and 2.5 volumes of ice-cold 95% ethanol. After 30 mins at −20° C., the DNA was pelleted by centrifugation at 13,000 rpm for 30 mins, washed with 70% ethanol and air-dried. The DNA pellet was resuspended in 100 μl 10 mM Tris/1 mM EDTA pH 8.0. For FCR, the following primers were used: sense, CGA GAA TTC ATG CTA ATG TTG CTA CGG; antisense, CGA AGC TTC TAG ATC TTC CTG CAT TGC. The cycling parameters were as follows: Initial denaturation at 94° C. for 3 mins, then 30 cycles at: 94° C. for 30 secs, 55° C. for 30 secs and 1 min at 68° C. A final extension at 68° C. was done for 10 mins. The PCR product was ligated into the T-vector pCR2.1 (Invitrogen), plasmid DNA was purified from recombinants and sequenced.

Observation: Sequencing revealed sequence ID NO:28, which is virtually identical to the Pid nucleotide sequence from plasmodium yoeli.

### REFERENCES

Aronov, A. M., Suresh, S., Buckner, F. S., van Voorhis, W. C., Verlinde, C. L. M. J., Opperdoes, F. R., Hol, W. G. and M. H. Gelb. (1999) Structure-based design of submicromolar biologically active inhibitors of trypanosomatid glyceraldehydes-3-phosphate dehydrogenase. Proc. Natl. Acad. Sci. 96 4273-4278
Breton, C. B., Blisnick, T., Jouin, H., Barale, J. C., Rabilloud, T., Langsley, G. & Da Silva, L. H. P. (1992) Plasmodium chabaudi p68 serine protease activity required for merozoite entry into mouse erythrocytes, Proc. Natl. Acad. Sci. USA 89,9647-9651
Burbelo, P. D., Drechel, D. & Hall, A. (1995) A conserved binding motif defines numerous candidate target proteins for Cdc42 and Rac GTPases, J. Biol. Chem. 270, 29071-29074
Burbelo, P. D., Snow, D. M., Bahou, W. & Spiegel, S. (1999) MSE55, a Cdc42 effector protein, induces long cellular extensions in fibroblasts, Proc. Natl. Acad. Sci. USA 96, 9083-9088.
Butler, D. & Maurice, J. (1997) Time to put malaria control on the agenda, Nature 386,535-541.
Chatfield, S. N., Strugnell, R. A., and G. Dougan. (1989) Live salmonellae as vaccines and carriers of foreign antigenic determinants. Vaccine, 7, 495-498.
Chen, C-Y. & Kado, C. I. (1994) Inhibition of oncogenicity of Agrobacterium tumefaciens by the osa gene of pSa, J. Bacteriol. 176,5697-5703.
Chen, L-M., Bobbie, S. & Galán, J. E. (1996) Requirement of CDC42 for Salmonella-induced cytoskeletal and nuclear responses, Science 274, 2115-2118.
Chien, C-H., Bartel, P. L., Sternglanz, R. and S. Fields. (1991) The two-hybrid system method to identify and clone genes for proteins that interact with a protein of interest. Proc. Natl. Acad. Sci. USA 88, 9578-9582
Combinatorial chemistry, (1996) Methods in Enzymology vol 267 ed. John N Abelson; Academic Press Inc. New York.
Gaillard, J.-L., Berche, P., Frehel, C., Gouin, E. & Cossart, P. (1991) Entry of L. monocytogenes into cells is mediated by internalin, a repeat protein reminiscent of surface antigens from Gram-positive cocci, Cell 65, 1127-1141.
Hadley, T. J. (1986) Invasion of erythrocytes by malaria parasites: a cellular and molecular overview, Annu. Rev. Microbiol. 40,451-477.
Hall, A. (1998) Rho GTPases and the actin cytoskeleton, Science 279, 509-514.
Hardt, W-D., Chen, L-M., Schuebel, K. E., Bustelo, X. R. & Galán, J. E. (1998) S. typhimurium encodes an activator of Rho GTPases that induces membrane ruffling and nuclear responses in host cells, Cell 93, 815-826.
Horuk, R., Chitnis, C. E., Darbonne, W. C., Colby, T. J., Rybicki, A., Hadley, T. J. & Miller, L. H. (1993) A receptor for the malarial parasite Plasmodium vivax: the erythrocyte chemokine receptor, Science 264,1182-1184.
Isberg, R. R. & Falkow, S. (1985) A single genetic locus encoded by Yersinia pseudotuberculosis permits invasion of cultured animal cells by Escherichia coli K-12, Nature 317, 262-264.
Jones, T. R., Obaldia, N. III, Gramsinski, R. A., Charoenvit, Y., Kolodny, N., Kitov, S., Davis, H. L., Krieg, A. M. and S. L. Hoffman. (1999) Synthetic oligonucleotides containing CpG motifs enhance immunogenicity of a peptide malaria vaccine in Aotus monkeys. Vaccine, 17, 3065-3071;
Lu, P-J., Zhou, X. Z., Shen, M., & Lu, K. P. (1999) Function of WW domains as phosphoserine- or phosphothreonine-binding modules, Science 283,1325-1328
Massol, P., Montcourrier, P., Guillemot, J. C. & Chavrier, P. (1998) Fc receptor-mediated phagocytosis requires CDC42 and Racl, EMBO J. 17, 6219-6229.
Mills, C. D., Burgess, D. C., Taylor, H. J. and K. C. Kain. (1999) Evaluation of a rapid and inexpensive dipstick assay for the diagnosis of Plasmodium falciparum malaria. Bull. WHO. 77, 553-559
Morris, G. E., thi Man, N., & Sedgwick, S. G. (1995) Epitope mapping of recombinant antigens by transposon mutagenesis, Mol. Biotech. 4, 45-54.
Parida, S. K., Domann, E., Rohde, M., Muller, S., Darji, A., Hain, T., Wehland, J. & Chakraborty, T. (1998) Internalin B is essential for adhesion and mediates the invasion of Listeria monocytogenes into human endothelial cells, Mol. Microbiol. 28, 81-93.
Ron, E., Turek, T. Mathiowitz, E., Chasin, M., MichaeL, H. and R. Langer (1993). Controlled release of polypeptides from polyanhydrides. Proc. Natl. Acad. Sci. USA 90, 4176-4180.
Sim, B. K. L., Chitnis, C. E., Wasniowska, K., Hadley, T. J., & Miller, L. H. (1994) Receptor and ligand domains for invasion of erythrocytes by Plasmodium falciparum, Science 264,1941-1944.
Sudol, M., Chen, H. I., Bougeret, C., Einbond, A. & Bork, P. (1995) Characterization of a novel protein-binding module—the WW domain, FEBS Lett 369, 67-71.
Susuki, T., Miki, H., Takenawa, T. & Sasakawa, C. (1998) Neural Wiskott-Aldrich syndrome protein is implicated in the actin-based motility of Shigella flexneri, EMBO J. 17, 2767-2776.
Valentine, C. R. I. & Kado, C. I. (1989) In Promiscuous plasmids of gram-negative bacteria (ed Thomas, C.) 125-163 (Academic Press, London,)
   Legends of Figures

FIG. 4 legends

A. Nucleic acid and translated amino acid sequence of pid and adjacent sequences. Upstream is ORF3, part of the osa operon in pSa; downstream of pid is the 5′ UTR of the antirestriction gene ArdC. Transposon insertion sites are indicated by arrow heads (□). Termination signals are indicated with asterisks. Sequences upstream of transposon insertion sites were obtained with the γ primer while downstream sequences were obtained with the δ primer of the Tn1000 transposon. Intervening sequences were obtained by primer-walking across the transposon insertion sites, and verified by sequencing several other clones including the wild-type cosmid construct Invcos18.

B. Schematic representation of pid as well as upstream and downstream sequences. The direction of transcription is indicated by an arrow. The putative Cdc42/Rho GTPase-interacting sequence (the CRIB domain) SEQ ID NO:6 is in bold print and is compared with that of Ste20/S cerevisiae (Acc # Lb4655); human WASP (Acc # NM000377); Cdc42 effector protein MSE55 (Acc # XM_{—}001058.1); p65^{PAK}-α/rat (Acc # NM017198); C09B8.7/C.elegans (Acc # U29612) and SHK1/S. pombe (Acc # AL034433).

1. An antigenic component, for use in a vaccine capable of promoting production in a subject of an antibody specific to the antigenic component, which antibody is capable of specifically binding to the Pid protein having the amino acid sequence in Seq ID No.1.

2. An antigenic component, for use in a vaccine capable of promoting production in a subject of an antibody specific to the antigenic component, which antibody is capable of specifically binding to the Pid protein having the amino acid sequence in Seq ID No.1, wherein the antigenic component comprises a component selected from the Pid protein having the amino acid sequence in Seq ID No 1 a peptide fragment of the Pid protein having the amino acid sequence in Seq ID No 1, and a variant of the Pid protein or peptide fragment thereof which does not substantially affect its antigenicity.

3. An antigenic component according to claim 2 which is preparable from an apicomplexan parasite.

4. An antigenic component according to claim 3 wherein the apicomplexan parasite is of a genus selected from the following :Eimeria; Isospora; Toxoplasma; Hammondia; Cystoisospora; Sarcocystis; Besnoitia; Frenkelia; Cryptosporidium; Plasmodium; Babesia; and Theileria.

5. An antigenic component according to claim 4 wherein the apicomplexan parasite is of the genus Plasmodium.

6. An immunogen comprising an antigenic component according to claim 1 coupled to an immunogenic component.

7. A vaccine comprising an immunogen according to claim 7 and an adjuvant.

8. A vaccine comprising a polynucleic acid, which encodes an antigenic component according to claim 1.

9. A vaccine according to claim 8 wherein the polynucleic acid further comprises a eukaryotic promoter for controlling expression of the sequence encoding the antigenic component.

10. A vaccine according to claim 8 which is suitable for use in a human subject.

11. A vaccine according to claim 10 which is suitable for use against human malaria caused by a parasite selected from: P. falciparum; P. ovale; P. vivax; and P. malariae.

12. A therapeutic agent comprising a component which component is capable of competing with a protein having the amino acid sequence in Seq ID No 1 in a specific binding assay.

13. A diagnostic agent comprising an antibody, which antibody is capable of specifically binding to the Pid protein having the amino acid sequence in Seq ID No 1.

14. A diagnostic agent comprising an antigenic component according to claim 1.

15. A pharmaceutical composition which comprises a protein comprising the amino acid sequence in SeqID No 1, or a peptide fragment thereof.

16. A pharmaceutical composition which comprises a polynucleic acid encoding a Pid protein having the amino acid sequence in SeqID No 1, or a fragment thereof.

17. A pharmaceutical comprising an antibody, which is capable of specifically binding to the Pid protein having the amino acid sequence in Seq ID No 1.

18. A method for prophylactic or therapeutic treatment of a disease caused by an apicomplexan parasite, which comprises administering to a subject an effective amount of a medicament comprising an antigenic component capable of promoting production in a subject of an antibody specific to the antigenic component, which antibody is capable of specifically binding to the Pid protein having the amino acid sequence in Seq ID No. 1.

19. A method for prophylactic or therapeutic treatment of a disease caused by an apicomplexan parasite, which comprises administering to a subject an effective amount of a medicament comprising an antigenic component capable of promoting production in a subject of an antibody specific to the antigenic component, which antibody is capable of specifically binding to the Pid protein having the amino acid sequence in Seq ID No. 1, wherein the antigenic component comprises a component selected from the Pid protein having the amino acid sequence in Seq ID No 1 a peptide fragment of the Pid protein having the amino acid sequence in Seq ID No 1, and a variant of the Pid protein or peptide fragment thereof which does not substantially affect its antigenicity.

20. A method according to claim 19 wherein the disease is selected from: malaria; coccidiosis; theileriosis; cryptosporidiosis; isosporiasis; blastocystosis; babesiosis; anaplasmosis; sarcosporidiosis; toxoplasmosis; and sarcocystosis.

21. A method according to claim 20 wherein the apicomplexan parasite is of the genus Plasmodium.

22. A method according to claim 21 wherein the apicomplexan parasite is one selected from the following: Plasmodium falciparum; Plasmodium vivax; Plasmodium ovale; and Plasmodium malariae.

23. A method for prophylactic or therapeutic treatment of malaria, which comprises administering to a human subject an effective amount of a vaccine comprising an antigenic component selected from the Pid protein having the amino acid sequence in Seq ID No 1 a peptide fragment of the Pid protein having the amino acid sequence in Seq ID No 1, and a variant of the Pid protein or peptide fragment thereof which does not substantially affect its antigenicity.

24. A method for diagnosing apicomplexan infection in a subject, which comprises:
(i) obtaining from the subject a sample of body fluid; and
(ii) testing the sample by contacting therewith an antibody capable of specifically binding to the Pid protein having the amino acid sequence in SeqID No 1.

25. A method for diagnosing apicomplexan infection in a subject, which comprises:
(i) obtaining from the subject a sample of body fluid; and
(ii) testing the sample by contacting therewith a protein comprising the amino acid sequence in SeqID No 1, or a peptide fragment thereof.

26. A method for diagnosing apicomplexan infection in a subject, which comprises:
(i) obtaining from the subject a sample of body fluid; and
(ii) testing the sample by contacting therewith a polynucleic acid encoding a Pid protein having the amino acid sequence in SeqID No 1, or a fragment thereof.

27. A method for prophylactic or therapeutic treatment of a disease caused by an apicomplexan parasite, which comprises administering to a subject an effective amount of a medicament comprising an inhibitor of Pid protein-Cdc42 interaction.

28. An in vitro method for diagnosing apicomplexan infection in a subject, which comprises:
(i) obtaining from the subject a nucleic acid containing sample; and
(ii) testing the sample for the presence of nucleic acid sequence characteristic of Pid.

29. A method according to claim 28, wherein the apicomplexan is of the genus Plasmodium.

30. A method according to claim 28, wherein sample comprises red blood cells.

31. A method according to claim 28, wherein the nucleic acid sample is amplified prior to testing.
